(19) United States
(12) Patent Application Publication
(10) Pub. No.: US 2026/0321396 A1
(43) Pub. Date: Oct. 15, 2026

(54) SYSTEMS, METHODS, AND DEVICES FOR MULTI-MODAL FUSION

(71) Applicant: PulseMedica Corp., Edmonton (CA)

(72) Inventors: Yevgeniy Eugene SHTEYN, Cupertino, CA (US); Christopher CEROICI, Edmonton (CA); Geoffroy RIVET-SABOURIN, Quebec City (CA); Nir KATCHINSKIY, Edmonton (CA)

(21) Appl. No.: 19/485,445

(22) PCT Filed: May 25, 2024
(86) PCT No.: PCT/IB2024/055106

§ 371 Date: Nov. 18, 2025

Related U.S. Application Data
(60) Provisional application No. 63/504,713, filed on May 26, 2023.

(51) Int. Cl. G16H 30/20; G06V 10/80; G16H 20/40; G16H 30/40

(52) U.S. Cl. CPC G16H 30/20; G06V 10/806; G16H 20/40; G16H 30/40

(57) ABSTRACT

An AI/ML model can generate latent vectors from input data received from data sources. A fusor can fuse the latent vectors to generate a fused latent vector. The fused latent vector can be used by a plurality of applications to perform a plurality of clinical tasks or functions. The fusor can be trained to automatically ignore redundant information. The AI/ML model may comprise encoders configured to generate the latent vectors. The encoders can be configured to ignore irrelevant information. The plurality of applications can each include a decoder configured to generate an output from a fused latent vector.

### CROSS-REFERENCE TO RELATED APPLICATIONS

This application claims the benefit of priority to U.S. Provisional Patent Application No. 63/504,713, filed May 26, 2023, the contents of which are hereby incorporated by reference in their entirety as if set forth fully herein.

### TECHNICAL FIELD

The present application relates to systems, methods, and devices for using latent vectors to capture, represent, store, and/or analyze medical data. Some implementations relate to the use of latent vectors for diagnosis, treatment, or both.

### BACKGROUND

The approaches described in this section are approaches that could be pursued, but not necessarily approaches that have been previously conceived or pursued. Thus, unless otherwise indicated, it should not be assumed that any of the material described in this section qualifies as prior art merely by virtue of its inclusion in this section.

Medical imaging offers many advantages. However, there are many challenges associated with medical imaging. Accordingly, there is a need for improved approaches to analyzing and using medical imaging data.

### SUMMARY

For purposes of this summary, certain aspects, advantages, and novel features are described herein. It is to be understood that not necessarily all such advantages may be achieved in accordance with any particular embodiment. Thus, for example, those skilled in the art will recognize the disclosures herein may be embodied or carried out in a manner that achieves one or more advantages taught herein without necessarily achieving other advantages as may be taught or suggested herein.

All of the embodiments described herein are intended to be within the scope of the present disclosure. These and other embodiments will be readily apparent to those skilled in the art from the following detailed description, having reference to the attached figures. The invention is not intended to be limited to any particular disclosed embodiment or embodiments.

Some embodiments herein relate to multimodal fusion of medical data. In some embodiments, multimodal data fusion refers to the process of integrating information from multiple sources or modalities into a single representation, in order to improve the accuracy, efficiency, completeness, or reliability of the resulting data. For example, in the field of computer vision, multimodal data fusion might involve combining information from visual, auditory, and other sensory inputs to identify objects in a scene. In some embodiments, multimodal data can be integrated from the same source but in different modes of operation (e.g., spatially or temporally). Similarly, in natural language processing, multimodal data fusion might involve integrating information from text, audio, and video to improve the accuracy of speech recognition or machine translation. In some embodiments, multimodal data fusion techniques can be used in a wide range of healthcare applications to assist healthcare providers and achieve superior results for patients. In some embodiments, multimodal fusion may leverage the strengths of different data sources to obtain a more complete and accurate understanding of a given problem or phenomenon.

One type of latent vector (LV) is an object-based LV, in which a separate LV is obtained for each object (e.g., a floater). Another type of LV is a patch-based LV, in which the information (e.g., image or volume) is divided into subsections, patch regions, or patches. In some embodiments, each patch can then be encoded to a latent vector and fused with latent vectors of other modalities in the same patch region. In some embodiments, a latent vector can be obtained for particular aspects of an object, such as pose or morphology.

In some embodiments, a modality that can be fused to the LV can be a biologically stimulated image. For example, a modality can be an image of a tissue or tissues with and without electrical stimulation. In another example, a patient interface designed to move or fixate the eye in a controlled fashion can enable imaging of floaters with and without eye motion to detection motion of structures.

As described herein, a modality is a specific type of data input, such as visual (for example, images and/or videos), auditory, textual (for example, doctor reports or lab results, or the like), and/or sensor data, among others. In some embodiments, multimodal data fusion involves combining information from two or more modalities to obtain a more complete or accurate representation of a given phenomenon. In some embodiments, several methods may be utilized to fuse multimodal data, including early fusion, late fusion, and hybrid fusion. Early fusion involves combining data at a low-level, while late fusion involves combining data at a high-level. Hybrid fusion combines elements of both early and late fusion.

In some embodiments, multimodal data fusion involves analyzing a database of medical data (e.g., images), automatically detecting data entry/sample modalities, automatically downloading/assigning encoders to generate LVs for data entries, and/or consolidating old/traditional databases to LV-based databases.

In some embodiments, multimodal data fusion provides data security for latent vectors by encrypting medical data. In some embodiments, LVs can only be interpreted/identified using decoders. In some embodiments, different security levels can be provided for different LVs. For example, a “complete” LV can be filtered to create “reduced” LVs that only contain information essential for a specific application. In some embodiments, data security is provided where an LV is used as a biological-based access/decryption key. In some embodiments, different levels of security can correspond to different levels of LV certainty. For example, a higher required security threshold can require a higher level of LV “certainty.”

In some embodiments, fusion can be performed on real-time or substantially real-Otime data. For example, in some embodiments, fusion can be performing on data streaming from an imaging device, such as an optical coherence tomography or scanning laser ophthalmoscopy imaging device. These are merely examples of devices, and other devices can be used additionally or alternatively, such as MRI scanners, x-ray scanners, CT scanners, ultrasound imagers, and so forth.

In some embodiments, multimodal data fusion has applications in a wide range of fields, multimodal data fusion can be used in healthcare to integrate information from electronic health records, medical images, and/or genetic data to develop personalized treatment plans for patients. In some embodiments, multimodal data fusion can be challenging due to differences in data format, noise, and/or missing data, among others. Additionally, selecting the most appropriate fusion method can be difficult, and may depend on the specific problems and data available. In some embodiments, the systems disclosed herein comprise a plurality of smart fusors, for example, a different smart fusor for each specific clinical situation, for example, detecting/tracking floaters during retinal surgery, or analyze ligaments or other tissue during knee surgery, or the like. In some embodiments, systems disclosed herein can be configured to store in an electronic database a plurality of fused latent vectors such that the systems provide an API (application programming interface) for allowing different software applications, some applications developed by third-parties, to interact, analyze, review, access, or connect with one or more of the plurality of fused latent vectors stored in the electronic database. In some embodiments, the software applications are made available to a user via an application store that allows the user to select different applications to apply to a selected fused latent vector data set, whereby different software applications reveal different analysis, results, recommendations, or the like to the user. In some embodiments, the systems disclosed herein can be configured to charge a fee to a user for access to one or more of the fused latent vector data sets and/or the one or more software applications configured to analyze a fused latent vector data set.

In some aspects, the techniques described herein relate to a computer-implemented method for fused latent vector processing, the computer-implemented method including: receiving, by a computer system, first data from a first data source; receiving, by the computer system, second data from a second data source; generating, via a first pretrained encoder, a first latent vector from the first data, wherein the first latent vector includes first information representing the first data; generating, via a second pretrained encoder, a second latent vector from the second data, wherein the second latent vector includes second information representing the second data; generating, via a pretrained fusor, a fused latent vector including fused information, wherein the fused information includes information representing the first information and the second information; and decoding the fused latent vector using a pretrained decoder, wherein the pretrained decoder is configured to generate an output for use in performing a clinical function.

In some aspects, the techniques described herein relate to a computer-implemented method, wherein the first data source includes image data from a first imaging device configured to capture image data using a first imagine device configure to capture image data using a first imaging modality, and wherein the second data source includes image data from a second imaging device configured to capture image data using a second imaging modality.

In some aspects, the techniques described herein relate to a computer-implemented method, wherein the first imaging device and the second imaging device are a same device, wherein the first imaging modality and the second imaging modality include a same imaging modality.

In some aspects, the techniques described herein relate to a computer-implemented method, wherein the first imaging modality and the second imaging modality include at least one of: optical coherence tomography (OCT), scanning laser ophthalmoscopy (SLO), ultrasound, computed tomography (CT), x-ray, magnetic resonance imaging (MRI), or fluorescein angiography (FA).

In some aspects, the techniques described herein relate to a computer-implemented method, wherein the pretrained fusor is configured to not incorporate redundant information in the fused information, wherein redundant information includes same information of the first information and the second information.

In some aspects, the techniques described herein relate to a computer-implemented method, further including: receiving, by the computer system, third data from a third data source; generating, via a third pretrained encoder, a third latent vector from the third data, wherein the third latent vector includes third information representing the third data; generating, via the pretrained fusor, a second fused latent vector including second fused information, wherein the pretrained fusor is configured to merge the fused latent vector and the third latent vector to generate the second fused latent vector.

In some aspects, the techniques described herein relate to a computer-implemented method, further including: determining a completeness of the second fused latent vector, wherein determining the completeness of the second fused latent vector includes: determining a similarity between the fused latent vector and the second fused latent vector; and comparing the similarity to a similarity threshold, wherein second fused latent vector is determined to be incomplete when the similarity is below the similarity threshold.

In some aspects, the techniques described herein relate to a computer-implemented method, wherein the first data includes a first OCT sample at a first location in a field of view, wherein the second data includes a second OCT sample at a second location in the field of view, and wherein the pretrained decoder is configured to generate an OCT image of the field of view.

In some aspects, the techniques described herein relate to a computer-implemented method, wherein the first data includes an OCT sample at a first location in a field of view, wherein the second data includes an SLO image of the field of view, and wherein the pretrained decoder is configured to generate an OCT image of the field of view.

In some aspects, the techniques described herein relate to a computer-implemented method, wherein the first data includes a first OCT sample at a first location in a field of view, wherein the second data includes a second OCT sample at a second location in the field of view, and wherein the pretrained decoder is configured to be used to generate a treatment plan, the treatment plan defining one or more treatment locations for treatment of a floater with a laser. In some embodiments, vitreous health can be a generalizing measure used to evaluate treatment options, outcomes, or both.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 shows a schematic of a system for multi-modal fusion according to some embodiments.

FIG. 2A shows a schematic of a model according to some embodiments.

FIG. 2B illustrates an example of a neural network according to some embodiments.

FIG. 3A shows a schematic of a system for training a model, encoder, decoder, and/or fusor according to some embodiments.

FIG. 3B shows a flowchart of a process for training a model, encoder, fusor, decoder, or any combination thereof according to some embodiments.

FIG. 4A illustrates an approach for training an encoder and/or fusor according to some embodiments.

FIG. 4B shows a schematic of a method for training a fusor to automatically determine redundant information according to some embodiments.

FIG. 5A shows a schematic of a method for determining completeness of a latent vector according to some embodiments.

FIG. 5B is a flowchart that illustrates a process for measuring change in a fused latent vector according to some embodiments.

FIG. 6 shows a schematic of an approach for determining latent vector completeness according to some embodiments.

FIG. 7 shows a schematic of a system for latent vector applications according to some embodiments.

FIG. 8 shows a schematic of a method for determining available applications for a latent vector or fused latent vector according to some embodiments.

FIG. 9 shows a schematic of a method for including additional information in a latent vector and/or a fused latent vector according to some embodiments.

FIG. 10 shows a schematic of a method for verifying a patient's identity according to some embodiments.

FIG. 11 shows a schematic of a method for generating a treatment plan according to some embodiments.

FIG. 12 shows a method and components of a system that can be used for performing a treatment procedure utilizing the approaches described herein according to some embodiments.

FIG. 13 illustrates a method and associated components of a process for using two-dimensional and three-dimensional data according to some embodiments.

FIG. 14 schematically illustrates a method for generating target-specific latent vectors according to some embodiments.

FIG. 15 schematically illustrates image reconstruction according to some embodiments.

FIG. 16 is a drawing that schematically illustrates combining two imaging modalities according to some embodiments.

FIG. 17A shows an example of sparse scanning according to some embodiments.

FIG. 17B schematically illustrates sparse scanning according to some embodiments, in which the sampling locations are selected based on a region of interest.

FIG. 18 is a flowchart that illustrates an example sparse scanning process according to some embodiments.

FIG. 19 shows examples of scanning paths according to some embodiments.

FIG. 20 is a drawing that schematically illustrates an example laser path and treatment points according to some embodiments.

FIG. 21 is a drawing that illustrates morphology and pose separation according to some embodiments.

FIG. 22 is a flowchart that illustrates an example process for generating pose and morphology latent vectors according to some embodiments.

FIG. 23 is a flowchart that illustrates an example process for treating a patient according to some embodiments.

FIG. 24 is a diagram that illustrates an example of generating and processing pose and morphology latent vectors according to some embodiments.

FIG. 25 illustrates an example of using morphology latent vectors and pose latent vectors according to some embodiments.

FIG. 26 shows an application for a pose latent vector according to some embodiments.

FIG. 27 illustrates a process for handling morphology latent vectors according to some embodiments.

FIG. 28 is a flowchart that illustrates an example process for updating identification confidence according to some embodiments.

FIG. 29 is a block diagram depicting an embodiment of a computer hardware system configured to run software for implementing one or more embodiments disclosed herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Vitreous floaters (“floaters) affect a significant portion of the population. In most cases, surgical intervention is not used to treat floaters. However, in severe cases where floaters significantly impact a patient, treatment may be warranted. Floaters can occur for a variety of reasons, such as posterior vitreous detachment, myopic vitreopathy, retinal tears, scleral buckling, vitreous hemorrhage, or retinopexy. Floaters can have significant impacts on lifestyle, work, and so forth. For example, patients who have severe problems with floaters may struggle with situations involving bright light, such as outdoor activities or working at a computer.

Vitrectomy is one option for improving vitreous health, for example by treating floaters. Vitrectomy is a surgical procedure in which a surgeon makes small incisions in the eye and removes the vitreous humor (or a portion thereof). However, this approach carries risk of infection, can accelerate cataract formation, and can result in retinal detachment. Laser treatment, for example with a yttrium aluminum garnet (YAG) laser, can reduce certain risks. Rather than removal of the vitreous humor, a floater can be broken up by exposure to a YAG laser. However, laser treatment can cause damage to the eye. Thus, it is important to apply treatment to target areas (e.g., to a floater) and limit exposure by other portions of the patient's eye. However, floaters are typically small and mobile, which can make targeting a laser difficult.

Described herein are approaches that can be used to enable improved floater screening, diagnosis, treatment, and post-treatment evaluation, as well as screening, diagnosis, treatment, and post-treatment evaluation of other medical conditions. However, the approaches herein are not limited to floater treatment or to medical applications but can be used in a wide variety of applications where it can be beneficial to combine multiple types of data to make decisions.

A healthcare provider (“provider”) may evaluate data from various sources, for example, the provider's notes, image data from one or more imaging modalities, and/or data from one or more other data sources to determine a diagnosis or treatment plan for a patient. In some cases, the determination of a diagnosis or treatment plan may be limited by the amount of information that the provider is able to process and analyze. The provider may ignore data from an imaging modality or data source based on provider preferences, training, or familiarity. Using traditional healthcare procedures, the provider may fail to detect information in image data, which may lead to the provider ordering unnecessary tests, imaging, and/or treatment that can cost patients and/or insurance significant amounts of money, time, and/or discomfort. The number of tests or imaging required for a provider to determine a screening or diagnosis or treatment plan can vary based on an experience level of the provider, which may lead to higher costs for certain patients.

As used herein, the term modality can refer to different imaging methods or other different sources of data. For example, one modality can be OCT imaging and another modality can be SLO imaging. However, modalities do not necessarily require different techniques or methodologies. For example, in some embodiments, a first modality can be an image sample taken in a first area and a second modality can be another image sample taken using the same equipment or technique in a second area. In some embodiments, different scanning patterns (e.g., raster, random, spiral, etc.) can be considered different modalities. In some embodiments, different imaging parameters (e.g., focus, capture time, etc.) can be considered different modalities.

In some embodiments, a computer system may analyze image data and/or data from one or more data sources. In some embodiments, the computer system may analyze or process new and/or redundant data. In some embodiments, the computer system may analyze data in real time or substantially real time, for example, during surgery. In some embodiments, processing redundant information may increase processing times and consume additional computing resources due to the computer system processing particular information multiple times. In some embodiments, the computer system may be trained to automatically identify redundant information and ignore the redundant information so the computer system does not process particular information multiple times.

In some embodiments, the computer system can be configured to analyze information from a single data source. In some embodiments, an artificial intelligence (AI) and/or machine learning (ML) model may be trained to analyze information from a single data source. In some embodiments, an AI/ML model may be trained to analyze information from a plurality of data sources.

In some embodiments, the systems described herein may comprise an AI and/or ML system configured to generate latent vectors for multiple imaging modalities and/or data sources. A latent vector can be a mathematical representation of an object or data point that captures important features or characteristics of that object or data point, such as the features indicative of a disease in an image, features unique to an individual (e.g., vein patterns), etc. Latent vectors can be generated by an encoder and/or fusor, which can learn to produce latent vectors for a variety of tasks such as classification, clustering, regression, and generative modeling. In some embodiments, latent vectors can be used in deep learning models such as neural networks. In some embodiments, latent vectors can be high-dimensional, meaning they have a large number of dimensions or features. In some embodiments, features may comprise abstract representations of the input data that capture important patterns or relationships. In some embodiments, latent vectors can be used for tasks such as image classification and clustering. For example, in image classification, a neural network may learn to generate a latent vector that captures important features of the input image such as edges, shapes, textures, and/or the like. In some embodiments, a latent vector can be used to classify the image into one of several categories. As another example, in a clustering application, a set of data points might be represented by their latent vectors and can be grouped into clusters based on their similarity in the latent space. In some embodiments, latent vectors may be used as tools for capturing complex relationships in medical data, which can enable a wide range of applications.

In some embodiments, latent vectors can represent image data and/or data from data sources. In some embodiments, the latent vectors may comprise an array of numbers representing image data and/or data from data sources. In some embodiments, the latent vector can be configured as a storage object of patient information. In some embodiments, a latent vector can represent image data from one or more images. In some embodiments, a latent vector can represent data from a single data source.

In some embodiments, a system for multimodal fusion may comprise one or more data sources. In some embodiments, the one or more data sources may include an imaging device, an imaging modality, a test result, a provider's notes, basic characteristics of a patient (e.g., sex, age, gender identity, height, weight, pre-existing conditions, etc.) and/or any other data source. In some embodiments, the imaging modality or imaging device may comprise scanning laser ophthalmoscopy (SLO), optical coherence tomography (OCT), x-ray, magnetic resonance imaging (MRI), computed tomography scan (CT), ultrasound, fluorescein angiography (FA), and/or any other imaging modality. In some embodiments, the test result may comprise results of a blood test, urinalysis, biopsy, mammogram, electroencephalogram (EEG), eye tests, and/or any other diagnostic test.

In some embodiments, the system may comprise a plurality of data sources. In some embodiments, the system may comprise a single source. In some embodiments, the system may comprise two data sources, three data sources, four data sources, five data sources, six data sources, seven data sources, eight data sources, nine data sources, ten data sources, eleven data sources, twelve data sources, thirteen data sources, fourteen data sources, fifteen data sources, sixteen data sources, seventeen data sources, eighteen data sources common nineteen data sources, twenty data sources, thirty data sources, forty data sources, fifty data sources, sixty data sources, seventy data sources, eighty data sources, ninety data sources, one hundred data sources, two hundred data sources, three hundred data sources, four hundred data sources, five hundred data sources, six hundred data sources, seven hundred data sources, eight hundred data sources, nine hundred data sources, one thousand data sources, two thousand data sources, three thousand data sources, four thousand data sources, five thousand data sources, six thousand data sources, seven thousand data sources, eight thousand data sources, nine thousand data sources, ten thousand data sources, one hundred thousand data sources. and/or any value between the aforementioned values.

In some embodiments, the data source may comprise a one-dimensional data source, for example, results of a blood test. In some embodiments, the data source may comprise a two-dimensional data source, for example, an SLO image. In some embodiments, the data source may include a three-dimensional data source, for example, a three-dimensional OCT image.

In some embodiments, the system may generate one or more outputs. In some embodiments, the one or more outputs may comprise a clinical determination, clinical function, clinical task, and/or a clinical decision. In some embodiments, the one or more outputs may comprise a reconstruction of input data, a diagnosis, a recommendation for one or more tests, a prognosis determination, instructions for a treatment device (e.g., a robotic treatment device), instructions for an imaging device (e.g., instructions for an imaging servo or galvanometer), an imagine in an image modality that was not provided, a treatment map, and/or any other clinical output. In some embodiments, the one or more outputs may comprise a one-dimensional output, such as, for example, a diagnosis, a two-dimensional output, such as, for example, an SLO image, and/or a three-dimensional output, such as, for example, a three-dimensional OCT image.

In embodiments, the system may comprise an AI and/or ML model. In some embodiments, the AI/ML model may comprise one or more elements. In some embodiments, the elements may comprise one or more encoders, fusors, and/or decoders, among others. In some embodiments, the AI/ML model may comprise an encoder for each data source. In some embodiments, the AI/ML model may comprise an encoder for each organ, appendage, and/or any other portion of a patient's body.

In some embodiments, an encoder is a type of neural network element that transforms input data from one or more data sources into a compressed representation, such as a latent vector, which captures important features of the original data. In some embodiments, the compressed representation, such as the latent vector, is typically used as input to another neural network component, such as a decoder, for further processing or output. In some embodiments, an encoder can be trained using unsupervised learning techniques, such as autoencoders, which involve training the network to reconstruct the original input from the compressed representation, or using supervised learning techniques where the encoder is trained to output specific labels or targets. In some embodiments, the encoder can be trained along with one or more decoders based on a desired decoder output. In some embodiments, the encoder can be trained with supervised learning when being trained to ignore redundant data, such as when aiming to make the LV the same as before and after the multimodal fusion.

In some embodiments, a fusor may be utilized in the process of combining multiple latent vectors (i.e., embedding vectors or representation vectors) into a single, aggregated latent vector (i.e., fused vector). In some embodiments, latent vectors are high-dimensional vectors that represent the features or characteristics of an input data point, such as an image or a sentence, in a lower-dimensional space. For example, in some embodiments, multiple convolutional neural networks (CNNs) or Transformer neural networks (TNN) can be used to extract different features from an image, and the resulting latent vectors can be combined to form a more robust and comprehensive representation of the image. In some embodiments, several methods may be used to fuse latent vectors, including simple averaging, weighted averaging, concatenation, or using more complex methods such as, for example, attention mechanisms. In some embodiments, the choice of fusion method depends on the specific application and the characteristics of the latent vectors being fused. In some embodiments, fusing latent vectors can help improve the performance and accuracy of the AI/ML model by incorporating multiple sources of information and creating more comprehensive representations of the input data.

In some embodiments, a decoder may comprise an element that transforms a compressed or encoded representation (e.g., a latent vector or fused latent vector) of data back into its original format. In some embodiments, the process of decoding is essentially the inverse of the encoding process and is sometimes referred to as “decoding” or “generating.” In some embodiments, a latent vector or fused latent vector of the input data is created using an encoder and/or fusor, and the decoder can be used to transform this representation back into the original format, such as an image, a sound wave, or a text sequence. In some embodiments, a decoder can be designed in various ways depending on the specific application and the type of data being decoded. For example, a decoder for image generation might use a convolutional neural network or transpose convolutional neural network architecture, while a decoder for text generation might use a recurrent or transformer neural network architecture.

In some embodiments, one or more encoders can be configured to generate a latent vector from input data received from the one or more data sources. In some embodiments, each encoder can be configured to generate a latent vector from input data captured by a data source of the one or more data sources. In some embodiments, the one or more encoders can be configured to generate a latent vector from input data comprising data in connection with a particular organ, appendage, and/or other portion of a patient's body. For example, a first encoder can be configured to generate latent vectors from image data of a retina and a second encoder can be configured to generate latent vectors from image data of a brain. In some embodiments, different encoders can be used to encode, for example, OCT images of a portion of an eye and SLO images of a portion of the eye. In some embodiments, each encoder of the one or more encoders can be configured to generate a latent vector from image data including a particular feature or indication of a condition. For example, a first encoder can be configured to generate latent vectors from image data including a floater and a second encoder can be configured to generate latent vectors from image data including a tumor. As another, first encoder can be configured to generate latent vectors from 3D image data (e.g., OCT data) including a floater, and the second encoder can be configured to generate latent vectors from 2D imaging data (e.g., SLO data) including the floater or an indication of the floater, such as a shadow. In some embodiments, different modalities can be encoded using the same encoder. For example, in the context where modalities are samples in different areas captured using the same imaging method, the same encoder can be used for latent vectors for each modality.

In some embodiments, the one or more encoders can automatically identify relevant information in the input data. In some embodiments, the one or more encoders can automatically determine whether particular information is relevant. In some embodiments, the one or more encoders can remove irrelevant information. In some embodiments, the one or more encoders may include only relevant information in the latent vectors. In some embodiments, the one or more encoders can be configured to generate a latent vector from a one-dimensional data source, a two-dimensional data source, and/or a three-dimensional data source. In some embodiments, the one or more encoders can be configured to generate a latent vector from a four-dimensional data source (e.g., three spatial dimensions and a time dimension).

In some embodiments, the AI/ML model may comprise a fusor. In some embodiments the fusor can be configured to fuse, merge, unify, join, or otherwise combine two or more latent vectors. In some embodiments, the fusor of the AI/ML model may generate a fused latent vector. In some embodiments, an input of the fusor may comprise two or more latent vectors. In some embodiments, the input of the fusor may comprise a latent vector and a pre-existing fused latent vector. In some embodiments, the fusor can fuse, merge, unify, join, or otherwise combine two or more fused latent vectors.

In some embodiments, latent vectors may have a size. In some embodiments, the size can be and/or can include a number of values in the latent vector. In some embodiments, the latent vector may comprise 1 value, 10 values, 100 values, 1000 values, 10,000 values, 100,000 values, 1 million values, 2 million values, 3 million values, 4 million values, 5 million values, 6 million values, 7 million values, 8 million values, 9 million values, 10 million values, and/or any value between the aforementioned values. In some embodiments, the latent vector may comprise more than 10 million values.

In some embodiments, the size of the latent vector or number of values in the latent vector can be based on an area or portion of the patient's body represented by the latent vector, and/or a disease or indication represented by the latent vector. In some embodiments, the size of the latent vector or number of values in the latent vector can be selected to comprise the encoders and/or the fusors.

In some embodiments, fused latent vector can have a size. In some embodiments, the size of the fused latent vectors may comprise a same size as the size of the latent vectors. In some embodiments, the fused latent vectors may comprise 1 value, 10 values, 100 values, 1,000 values, 10,000 values, 100,000 values, 1 million values, 2 million values, 3 million values, 4 million values, 5 million values, 6 million values, 7 million values, 8 million values, 9 million values, 10 million values, and/or any value between the aforementioned values.

In some embodiments, the AI/ML model may comprise one or more decoders. In some embodiments, the decoders may be configured to generate an output. In some embodiments, the decoders may be configured to generate an output from information in a latent vector and/or a fused latent vector. In some embodiments, each decoder may be configured to generate a particular output.

In some embodiments, the encoders, the fusors, and/or the decoders may comprise a neural network. In some embodiments, the neural network may comprise a deep learning neural network, backpropagation neural network, feedforward network, recurrent network, convolutional network, and/or any other type of neural network. In some embodiments, a neural network comprises a type of ML model that comprises interconnected nodes, called neurons, which are organized in layers. In some embodiments, each neuron receives input from other neurons or from external sources, and processes that input to produce an output, which is then passed on to other neurons in the next layer. In some embodiments, a neural network is trained on a large set of input data, with the goal of learning to recognize patterns and make accurate predictions based on that data.

In some embodiments, the neural network may comprise input nodes, hidden nodes, and/or output nodes. In some embodiments, input nodes may receive input data from one or more data sources. The input nodes can pass their outputs on to the next layer of neurons, which may generally comprise hidden nodes. In some embodiments, hidden nodes perform computations on the input data, and their output is passed on to other hidden nodes or to output nodes. In some embodiments, output nodes comprise the final layer of neurons in the neural network and produce the output of the neural network. In some embodiments, the output may comprise a classification, a prediction, or a decision. In some embodiments, the number of input nodes and output nodes may be fixed based on the nature and amount of input and output data. In some embodiments, the number of hidden nodes may be adjusted to improve the performance of the network, depending on the complexity of the task, the size of the training data, and/or the quality of the training data. In some embodiments, non-linear transformation layers (also referred to herein as activation functions) can be included in the neural network.

In some embodiments, the input nodes can receive input data. In some embodiments, the input nodes can transmit the input data to the plurality of hidden nodes. In some embodiments, the input nodes can produce an intermediate output that is passed to one or more hidden nodes. In some embodiments, plurality of hidden nodes can each perform one or more calculations. In some embodiments, the plurality of hidden nodes can each transmit an output of the one or more calculations to one or more other hidden nodes. In some embodiments, the plurality of hidden nodes can transmit the output of the one or more calculations to the output nodes. In some embodiments, the output nodes can generate output data of the neural network. In some embodiments, the output data of the neural network may comprise a latent vector, a fused latent vector, a clinical function or task, and/or any other information or output described herein.

In some embodiments, a user may construct a neural network by defining one or more parameters of the network. For example, in some embodiments, the user may define a number of hidden nodes in the neural network. In some embodiments, the user may define or determine one or more connections between each hidden node. In some embodiments, the user may select a number of input nodes and/or output nodes.

In some embodiments, the neural network may comprise one or more mathematical models, including, but not limited to models based on linear algebra, calculus, activation functions, weighted sums, backpropagation, convolution, and/or recursion, among others. In some embodiments, the user can select one or more portions of the one or more mathematical models to include in the neural network.

In some embodiments, the AI/ML model, the encoder, the fusor, and/or the decoder can be trained. In some embodiments, fused latent vectors generated by the fusor can constrain the encoders and/or the decoders during training so latent vectors generated by the encoders and/or outputs generated by the decoders depend on information included in the fused latent vector by the fusor. In some embodiments, the encoders, the fusor, and the decoders can be trained at the same time. In some embodiments, the AI/ML model, which may comprise a neural network, may be trained using backpropagation, which is an optimization algorithm that adjusts the weights of the network to minimize the error between the predicted output and the actual output. In some embodiments, the training process can include one or more of the following operations: initialization, forward pass, error computation, backward pass, and/or weighting updates. In some embodiments, the training process can include repeating one or more of the operations for multiple iterations until the network converges to a set of weights that minimize error when using the training data. In some embodiments, the performance of the trained network may be evaluated on a separate validation set of data to avoid overfitting of the training data.

In some embodiments, the AI/ML model can transmit input data to the encoders. In some embodiments, the input data can be captured by one or more data sources. In some embodiments, the encoders can each generate a latent vector including information representing input data from a data source. In some embodiments, the fusor can fuse, merge, unify, join, or otherwise combine each latent vector to generate a fused latent vector.

In some embodiments, each decoder can generate an output from the fused latent vector. In some embodiments, each output may comprise a reconstruction of the input data received from each data source. In some embodiments, the AI/ML model can compare each output to a desired output. In some embodiments, the desired output may comprise corresponding input data from each data source. In some embodiments, the system can compare each output to a desired output to determine an error between the output and the desired output. In some embodiments, if the desired output is a reconstruction of the input data, the error may comprise a percentage or other numerical representation of the difference between the output data and the input data. In some embodiments, if the desired output is a diagnosis, identification of one or more features in the input data, a recommendation for one or more tests to perform, a prognosis determination, and/or any other clinical determination or decision, the error may comprise whether the output is a same output as the desired output.

In some embodiments, the error may be measured using a loss function, also called a cost function or objective function. In some embodiments, a loss function measures the difference between the predicted output of the model and the actual output, for a given input example. In some embodiments, a mean squared error (MSE) loss function may be used, which measures the average squared difference between the predicted and actual output values. In some embodiments, training a neural network minimizes the value of the loss function over the training examples, by adjusting the weights of the network.

In some embodiments, based on the error, the system and/or the AI/ML model can automatically modify or adjust the encoders, the fusor, the decoders, or any combination thereof. In some embodiments, the system and/or the AI/ML model can modify or adjust the encoders, the fusor, the decoders, or any combination thereof to reduce, minimize, or eliminate the error.

In some embodiments, after the AI/ML model modifies or adjusts the encoders, the fusor, and/or the decoders, the AI/ML model can repeat the training with same input data or different input data. In some embodiments, the system can repeat the training a plurality of iterations until the error is below a predetermined error threshold. In some embodiments the AI/ML model can repeat the training a plurality of iterations until the error is reduced, minimized, or eliminated. In some embodiments, a training iteration comprises a single update of the network's weights using one or more training examples. In some embodiments, during each iteration, the network processes a batch of training examples, computes the error, and updates the weights using, for example, an optimization algorithm. In some embodiments, the number of iterations required to train a neural network depends on several factors, including the size and complexity of the network, the size and quality of the training data, and the optimization algorithm used. In some embodiments, during each iteration, the network adjusts its weights in the direction that minimizes the error, with the goal of improving its performance on the training data. In some embodiments, as the network's weights are updated, the predictions of the network become more accurate, and the error decreases.

In some embodiments, in each training iteration, the encoders can automatically determine which information to include in latent vectors, so the latent vectors produce outputs with the smallest error. In some embodiments, in each training iteration, the fusor can automatically determine which information to include in fused latent vectors, so the fused latent vector produces outputs with the smallest error. In some embodiments, each time the AI/ML model repeats the training, the decoders can automatically determine which information in a fused latent vector to use to generate that particular output. In some embodiments, the AI/ML model may be trained for a fixed number of iterations or until a condition is met, such as improvement in a loss function dropping below a threshold value. In some embodiments, the AI/ML model may be trained until improvement in performance of the model (e.g., improvement in the loss function) decreases below a threshold value. IN some embodiments, the AI/ML model may be trained until performance begins to decline when the model is tested using validation data or other data other than the training data used to train the model.

In some embodiments, since the encoders, the fusor, the decoders, or any combination thereof are trained at a same time, new encoders configured to generate latent vectors from a new data source can be added to the AI/ML model, and the fusor can be retrained to include latent vectors generated from the new data source in use the latent vectors. In some embodiments, additional decoders configured to generate new outputs can be added to the AI/ML model, and the fusor can be retrained to include information in the fused latent vectors needed to generate the new outputs.

In some embodiments, the AI/ML model can be trained to automatically determine redundant information in latent vectors. In some embodiments, the fusor can be trained to automatically determine redundant information in latent vectors. In some embodiments, the AI/ML model can compare fused latent vectors generated by the fusor to the latent vectors used to generate the fused latent vectors. In some embodiments, AI/ML model can penalize the fusor each time the fusor includes redundant information in a fused latent vector so the fusor can automatically and/or dynamically learn to identify redundant information. In some embodiments, redundant information may comprise information in a latent vector that is already in another latent vector and/or a fused latent vector.

In some embodiments, the fusor can receive an indication of which information is redundant information. In some embodiments, the fusor can use the indication of which information is redundant information to automatically learn which information to include in fused latent vectors. In some embodiments, based on the indication of which information is redundant information, the AI/ML model can automatically disregard redundant information in the resulting latent vector.

In some embodiments, the AI/ML model can automatically determine if a latent vector is complete. In some embodiments, the fusor can generate a fused latent vector from a first latent vector and a second latent vector. In some embodiments, the first latent vector and the second latent vector can include latent vectors generated from input data captured at different times. In some embodiments, the first latent vector and the second latent vector can include latent vectors generated from input data captured at the same time. In some embodiments, the first latent vector and the second latent vector can include latent vectors generated from input data captured from a same data source. In some embodiments, the first latent vector and the second latent vector can include latent vectors generated from input data captured from different data sources. In some embodiments, the AI/ML model and/or the fusor can determine a change between the first latent vector and the fused latent vector. In some embodiments, the change can include an amount of information in the fused latent vector that is not in the first latent vector. In some embodiments, the AI/ML model can compare the change to a predetermined change threshold. In some embodiments, the predetermined change threshold can be based on a clinical function or task that the fused latent vector will be used for. For example, if the fused latent vector will be used for diagnosis, the predetermined change threshold may be higher than if the clinical function or task the fuse latent vector will be used for is generating instructions for a surgical device. For example, when used for diagnosis, less complete information may still be acceptable, as diagnostic decisions are ultimately made by a healthcare provider after human review. However, instructions for a surgical device may be more sensitive to errors or incomplete information which could result in, for example, treatment being performed in an incorrect location. In some embodiments, a human may adjust the change threshold.

In some embodiments, if the change is below the predetermined change threshold, the AI/ML model can determine that the fused latent vector is complete. In some embodiments, if the change is above the predetermined change threshold, the AI/ML model can determine that the fused latent vector is incomplete.

In some embodiments, the AI/ML model can determine a total amount information in the fused latent vector. In some embodiments, the AI/ML model can fuse multiple latent vectors with the fused latent vector. In some embodiments, each time the AI/ML model fuses a latent vector with the fused latent vector, the AI/ML model can determine the change. In some embodiments, the AI/ML model can sum each change to determine the total amount of information in the fused latent vector. In some embodiments, the AI/ML model can determine a completeness of the fused latent vector by comparing the total amount of information in the fused latent vector to a size of the fused latent vector or a maximum amount of information the fused latent vector is configured to hold, store, or contain. In some embodiments, the completeness of the fused latent vector can include a percentage or other numerical representation of the total amount of information in the fused latent vector compared to the size of the fused latent vector or the maximum amount of information the fused latent vector is configured to hold, store, or contain. In some embodiments, the AI/ML model can compare the completeness of the fused latent vector to a predetermined completeness threshold. In some embodiments, if the completeness of the fused latent vector is above the predetermined completeness threshold, the AI/ML model can determine that the fused latent vector is complete. In some embodiments, if the completeness of the fused latent vector is below the predetermined completeness threshold, the AI/ML model can determine that the fused latent vector is incomplete. In some embodiments, completeness determinations can be based on indications of the amount of information stored in the fused latent vector. Such indications can generally indicate a level of completeness but may not be an exact measure of completeness.

In some embodiments, if the fused latent vector is complete, the AI/ML model can confirm that the fused latent vector is complete. In some embodiments, the AI/ML model can fuse multiple latent vectors with the fused latent vector after the AI/ML model determines the latent vector is complete. In some embodiments, the AI/ML model can determine if the latent vector is complete each time the AI/ML model fuses a latent vector with the fused latent vector to confirm the fused latent vector is complete.

In some embodiments, if the fused latent vector is incomplete, the AI/ML model can request or suggest additional information and receive additional input data. In some embodiments, an encoder can generate a latent vector from the additional input data, and the fusor can fuse the latent vector from the additional input data with the fused latent vector.

In some embodiments, the AI/ML model can determine latent vector completeness using real time or substantially real time input data. In some embodiments, if the AI/ML model determines that the fused latent vector is complete, the AI/ML model can continue to receive input data for a period of time after the AI/ML model determines that the fused latent vector is complete. In this way, the AI/ML model can ensure that the fused latent vector includes sufficient information.

In some embodiments, systems described herein may use latent vectors for a plurality of applications. In some embodiments, the latent vectors may comprise an information or file type, for example, the latent vector may comprise a file type for the system.

In some embodiments, the system may comprise a latent vector interface. In some embodiments, a user, such as a provider, can access a latent vector database via the latent vector interface. In some embodiments, the latent vector interface may comprise an application programming interface (API) to allow a user and/or a user device to transmit and/or access information in the latent vector database. In some embodiments, the user can access the latent vector database via a user device, such as a personal computer, cellular phone, smartphone, laptop, tablet computer, and/or any other computing device. In some embodiments, the user can access the latent vector database via a diagnostic or screening device, a surgical device, and/or any other medical device.

In some embodiments, the latent vector database may comprise a plurality of latent vectors. In some embodiments the plurality of latent vectors may comprise latent vectors for one or more organs, appendages, and/or other portions of a patient's body. In some embodiments, the latent vector database may comprise one or more latent vectors for a plurality of patients.

In some embodiments, the system may comprise a plurality of applications. In some embodiments, each application may comprise an encoder. In some embodiments, each application can be configured to generate a particular output from a latent vector. In some embodiments, each application can be configured to generate an output from latent vectors representing particular organs, appendages any other portion of a patient's body, indications of a condition, etc.

In some embodiments, new applications can be added to the system, and the new applications can function with a plurality of different data sources, as each application may be configured to generate outputs from latent vectors. In some embodiments, new data sources can be utilized by the system, and the applications may be configured to function with the new data sources, as the input data from the new data sources can be represented by a latent vector.

In some embodiments, the AI/ML model may be utilized to verify a patient's identity. In some embodiments, an encoder may generate a latent vector from input data captured from a patient in real time or substantially real time. In some embodiments, the AI/ML model can compare the latent vector from input data captured from the patient in real time or substantially real time to a latent vector from the latent vector database that corresponds to the patient to determine if the system is using correct latent vectors. In some embodiments the AI/ML model can verify a patient's identity to, for example, ensure that a correct patient is in an operating room or procedure room for a surgical or other treatment procedure. In some embodiments the AI/ML model can verify a patient's identity to ensure a provider performs a correct operation, treatment, or surgical procedure on a patient. In some embodiments, identity verification can be based on biometric information, such as ocular imaging.

In some embodiments, the AI/ML model may be used in real time or substantially real time during surgery. In some embodiments, the AI/ML model may receive input data from a data source at a first time. In some embodiments, an encoder can generate a latent vector from the input data. In some embodiments, the AI/ML model can receive second input data from the data source at a second time. In some embodiments, the encoder can generate a second latent vector from the second input data. In some embodiments, a fusor can fuse, merge, unify, join, or otherwise combine the latent vector and the second latent vector to generate a first fused latent vector. In some embodiments, the AI/ML model can transmit the first fused latent vector to an application. In some embodiments, an application can be notified that a new fused latent vector is available, and the application can retrieve the new fused latent vector from a data store.

In some embodiments, the AI/ML model can receive third input data from the data source at a third time. In some embodiments, the encoder can generate a third latent vector from the third input data. In some embodiments, the fusor can fuse, merge, unify, join, or otherwise combine the first fused latent vector and the third latent vector to generate a second fused latent vector. In some embodiments, the AI/ML model can transmit the second fused latent vector to the application.

It will be appreciated that the number of latent vectors and the number of fused latent vectors that can be generated is not necessarily limited.

In some embodiments, the input data received at a first time may comprise input data from an OCT scanner. In some embodiments, the input data received at the second time and/or the third time may comprise input data from an SLO scanner. In some embodiments, the application can be configured to generate OCT images from the first fused latent vector and/or the second fused latent vector, wherein the OCT images represent what an OCT image would look like at the second time and/or the third time. This can be advantageous because, for example, SLO images can generally be captured more quickly than OCT images.

In some embodiments, the AI/ML model can segment input data into a plurality of segments. In some embodiments, the encoders can be configured to generate latent vectors for a particular segment of the plurality of segments. In some embodiments, each segment may comprise a target. In some embodiments, the target may comprise a feature or indication of a condition that is being tracked during a treatment procedure. In some embodiments, the system can generate a fused latent vector from latent vectors captured for each segment such that each segment includes a corresponding fused latent vector.

In some embodiments, generating latent vectors from input data from a plurality of different data sources can consolidate patient data or information. In some embodiments, generating latent vectors from input data from a plurality of different data sources can make connections between patient outcomes to particular tests, analysis, and/or surgical procedures. In some embodiments, the AI/ML model can quantify treatment planning and best practices based on previous patient outcomes and incorporate analysis of previous patient outcomes into recommendations for future patients. In some embodiments, the AI/ML model and/or latent vectors can reduce or eliminate redundant, repetitive, or unnecessary testing. In some embodiments, the AI/ML and/or latent vectors can lower treatment costs for patients and/or insurance companies. In some embodiments, the AI/ML model may provide justification for the administration of certain tests and/or diagnostic/imaging procedures based on an analysis of fused latent vectors in order to determine missing patient data that the fusor or another component of the systems and methods described herein recommends. In some embodiments, these recommendations may be based on an analysis of historical data that suggests that the missing data is clinically important for achieving better patient outcomes.

### Multi-Modal Fusion

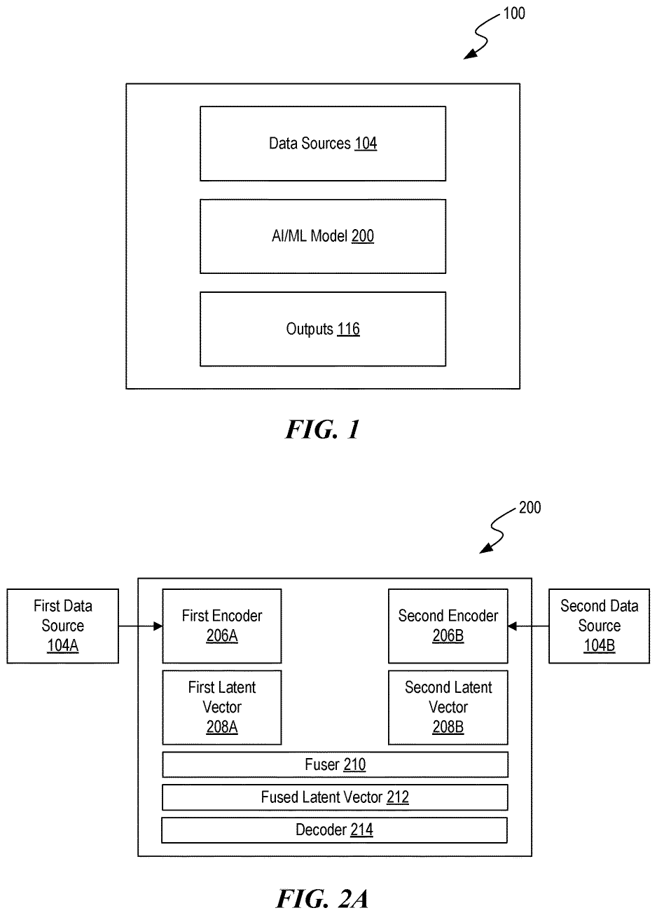

FIG. 1 shows a schematic of a system 100 for multi-modal fusion. In some embodiments, the system 100 may comprise data sources 104. In some embodiments, the data sources 104 can comprise a single data source. In some embodiments, the data sources 104 can comprise a plurality of data sources.

In some embodiments, the data source 104 may comprise an image for an imaging modality, a test result, a clinician's notes, and/or any other type of data. In some embodiments, the imaging modality may comprise scanning laser ophthalmoscopy (SLO), optical coherence tomography (OCT), x-ray, magnetic resonance imaging (MRI), computed tomography (CT) imaging, ultrasound, fluorescein angiography (FA), and/or any other imaging modality. In some embodiments, a modality can be defined in a different manner, for example as a segment or portion of an imaging scan. For example, in some embodiments, a modality can be an OCT scan in a specific region of a larger area. In some embodiments, the test result may comprise results of a blood test, urinalysis, biopsy, mammogram, electroencephalogram (EEG), eye tests, and/or any other diagnostic test.

In some embodiments, the data source 104 may comprise a one-dimensional data source 104, for example, results of a blood test. In some embodiments, the data source 104 may comprise a two-dimensional data source 104, for example, an SLO image. In some embodiments, the data source 104 may comprise a three-dimensional data source 104, for example, a three-dimensional OCT image. In some embodiments, the data source 104 can include a four-dimensional data source 104, for example, a three-dimensional image with a time component. In some embodiments, a data source can be configured to store a plurality of 1D, 2D, and/or 3D data. In some embodiments, there can be a time component (e.g., a timestamp) associated with the data, which can enable time-dependent visualization, analysis, etc. 3D data can include, for example, position (e.g., x, y, z) data, rotational data, etc. In some embodiments, a data source can include 6D data, such as the combination of 3D position and 3D rotation. In some embodiments, a data source can include 7D data, such as a combination of 3D position, 3D rotation, and a time component.

In some embodiments, the system 100 may comprise or produce outputs 116. In some embodiments, the outputs 116 may comprise a reconstruction of the input data, a diagnosis, a recommendation for one or more tests to perform, a prognosis determination, and/or any other clinical determination or decision.

In some embodiments, the outputs 116 may comprise a one-dimensional output 116, such as, a diagnosis, a two-dimensional output 116, such as, an SLO image, and/or a three-dimensional output 116, such as, a three-dimensional OCT image.

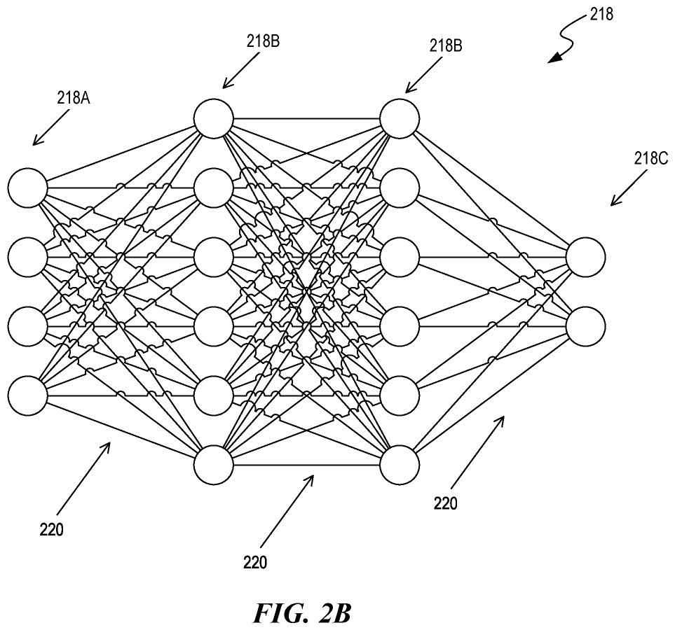

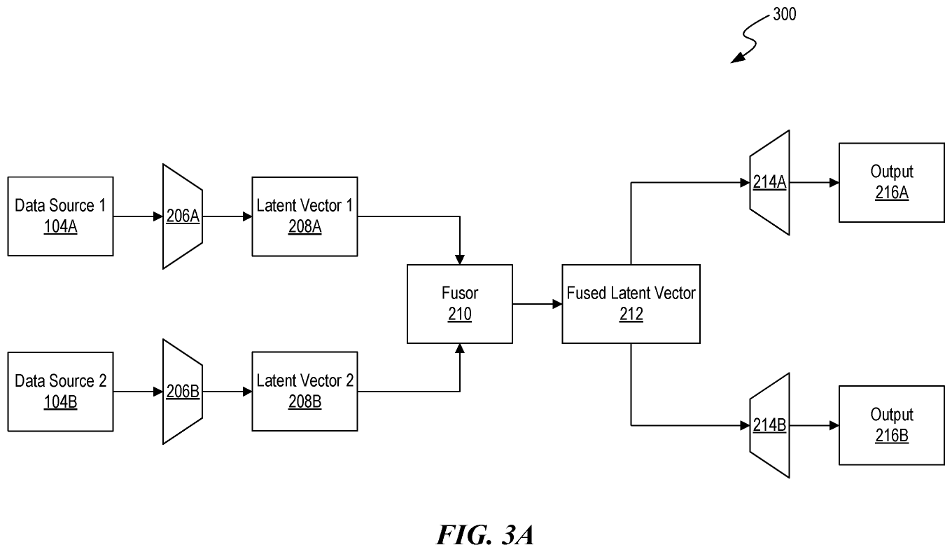

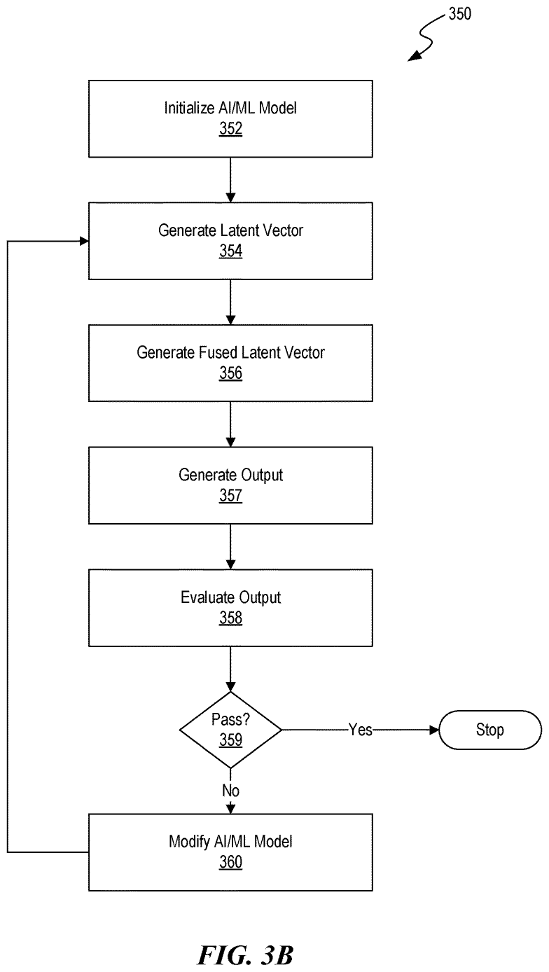

In some embodiments, the system 100 may comprise a model 200 (e.g., an AI/ML model). FIG. 2A shows a schematic of the model 200. In some embodiments, the model 200 may comprise a first encoder 206A and a second encoder 206B (collectively, encoders 206). In some embodiments, the model 200 may comprise an encoder for each data source (e.g., data source 104). In some embodiments, the encoders 206 can be configured to generate a latent vector 208A and a latent vector 208B (collectively, latent vectors 208) from input data received from the data source 104A and the data source 104B (collectively, data sources 104). In some embodiments, as further described below with reference to FIGS. 3A-3B, each encoder 206 can be configured or trained to generate a latent vector 208. In some embodiments, each encoder 206 can generate a latent vector 208 from input data received from a specific data source 104. For example, a first encoder 206A can be configured to generate a first latent vector 208A from input data received from a first data source 104A, and a second encoder 206B can be configured to generate a second latent vector 208B from input data received from a second data source 104B.

In some embodiments, the encoders 206 can generate latent vectors 208 for a particular part of a patient's body. For example, an encoder 206A can be configured to generate latent vectors 208A from image data of a retina, and another encoder 206B can be configured to generate latent vectors 208B from image data of a brain.

In some embodiments, the encoders 206 can generate latent vectors 208 for a particular feature of the particular part of the patient's body. For example, an encoder 206 can be configured to generate latent vectors 208 from image data including a floater, and another encoder 206 can be configured to generate latent vectors 208 from image data including a tumor.

In some embodiments, the encoders 206 can automatically determine relevant information in the input data. In some embodiments, the encoders 206 can automatically determine information that is not relevant, and the encoders 206 can remove the information that is not relevant from the input data when the encoders 206 generate the latent vectors 208. For example, if the encoder 206 is configured to generate latent vectors 208 that represent a floater in a retina, the relevant information may comprise image data of the floater and the information that is not relevant may comprise image data of the structure of the eye.

In some embodiments, the encoder 206 can automatically generate a latent vector 208 that may comprise the relevant information (e.g., only the relevant information or substantially only the relevant information). In some embodiments, the encoder 206 can generate a latent vector 208 from the image data and the encoder 206 can automatically remove information that is not relevant.

In some embodiments, the encoder 206 can be configured to generate a latent vector 208 from a one-dimensional data source 104, a two-dimensional data source 104, a three-dimensional data source 104, and/or a four-dimensional data source (e.g., three spatial dimensions and a time dimension). In some embodiments, the data can have more than four dimensions, such as six dimension (e.g., to represent spatial and rotational coordinates) or seven dimensions (e.g., to represent spatial coordinates, rotational coordinates, and time).

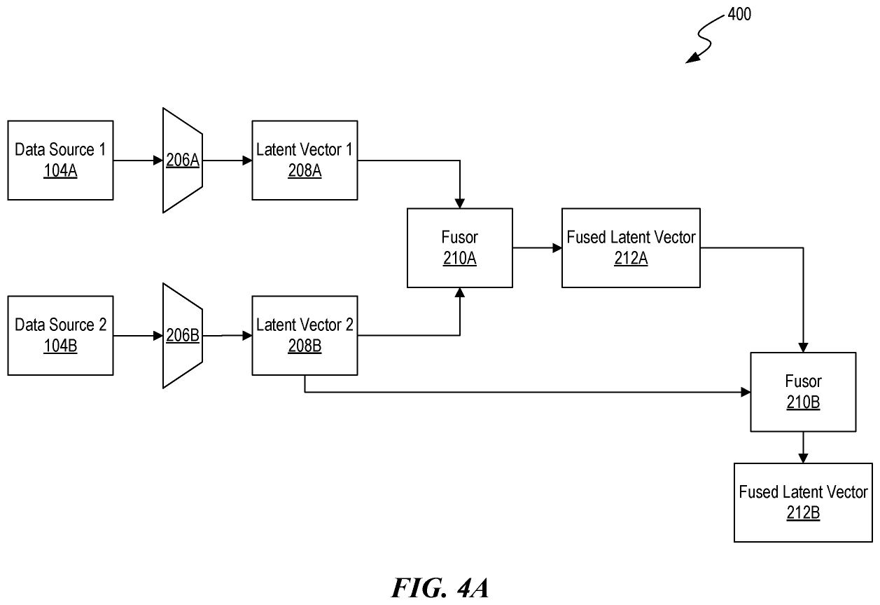

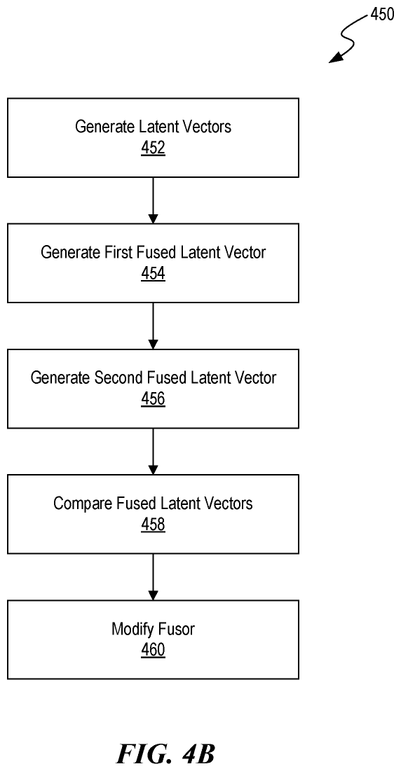

In some embodiments, the model 200 may comprise a fusor 210. In some embodiments, the fusor 210, as further described below with reference to FIGS. 3A-4B can be configured to generate a fused latent vector 212. In some embodiments, the fusor 210 can be configured to fuse, merge, unify, join, or otherwise combine two or more latent vectors 208. In some embodiments, the fusor 210 can comprise a transformer. In some embodiments, the fusor 210 can be configured to fuse, merge, unify, join, or otherwise combine two or more latent vectors 208 wherein each latent vector 208 represents information from a different data source 104. In some embodiments, the fusor 210 can be configured to fuse, merge, unify, join, or otherwise combine two or more latent vectors 208 wherein each latent vector 208 represents information from a same data source 104. In some embodiments, the fusor 210 can be configured to fuse, merge, unify, join, or otherwise combine a first latent vector 208A from a first data source 104A and a second latent vector 208B from a second data source 104B.

In some embodiments, an input of the fusor 210 may comprise two or more latent vectors 208. In some embodiments, the input of the fusor 210 may comprise a latent vector 208 and a fused latent vector 212. In some embodiments, the input of the fusor 210 may comprise two or more fused latent vectors 212. In some embodiments, the input of the fusor 210 may comprise one or more latent vectors 208 and a one or more fused latent vectors 212.

In some embodiments, the latent vector 208 may comprise a size (e.g., a number of features). In some embodiments, the size may comprise a number of values in the latent vector 208. In some embodiments, the latent vector 208 may comprise one value or a plurality of values.

In some embodiments, the size of the latent vector 208 or number of values in the latent vector 208 can be based on an area or portion of a patient's body represented by the latent vector 208, a disease or indication represented by the latent vector 208, a resolution of an image used to generate the latent vector, and so forth.

In some embodiments, the fused latent vector 212 may comprise a size. In some embodiments, the size of the fused latent vector 212 may comprise a same size as the size of the latent vector 208. In some embodiments, the size of the fused latent vector 212 may comprise a number of values in the fused latent vector 212. In some embodiments, the size of the fused latent vector may comprise a same size as the sum of the sizes of one or more latent vectors, fused latent vectors, or both used to generate the fused latent vector 212. In some embodiments, the fused latent vector 212 may comprise a single value or multiple values. The size of a fused latent vector is not necessarily linked to the size of the latent vectors used in generating the fused latent vector. In some embodiments, the size of a fused latent vector can be fixed, such that the size of the fused latent vector does not change as additional latent vectors are fused to the fused latent vector. Thus, it will be appreciating that a fusing operation is not necessarily a simple concatenation of latent vectors. Rather, in some embodiments, values in a fused latent vector can be updated to incorporate additional information obtained from additional latent vectors.

In some embodiments, the model 200 may comprise a decoder 214. In some embodiments, the model 200 can include a plurality of decoders. In some embodiments, the decoder 214 can be configured to generate an output 116 from a latent vector 208 and/or a fused latent vector 212. In some embodiments, the decoder 214 can be configured to generate an output 116 from a plurality of latent vectors 208, a plurality of fused latent vectors 212, or both.

In some embodiments, as further described below with reference to FIG. 3A-3B, the decoder 214 can be configured or trained to determine information automatically and/or dynamically in a latent vector 208 and/or a fused latent vector 212 that is relevant for the output 116.

In some embodiments, the encoder 206, the fusor 210, the decoder 214, or any combination thereof may comprise a neural network 218, as shown in FIG. 2B. In some embodiments, the neural network 218 may comprise a deep learning neural network, a backpropagation neural network, both, or any other suitable network or combination of networks. In some embodiments, the neural network 218 may comprise input nodes 218A. In some embodiments, the neural network 218 may comprise a plurality of hidden nodes 218B. In some embodiments, neural network 218 may comprise output nodes 218C. In some embodiments the input nodes 218A can receive input data. In some embodiments, the input nodes 218A can transmit the input data to the plurality of hidden nodes 218B or can process the input data and output the results of the processing to the hidden nodes 218B, e.g., to a first layer of the hidden nodes 218B. In some embodiments, the plurality of hidden nodes 218B can each perform one or more calculations. In some embodiments, the plurality of hidden nodes 218B can each transmit an output of the one or more calculations to a plurality of other hidden nodes 218B, for example from a first layer of hidden nodes to a second layer of hidden nodes. In some embodiments, the plurality of hidden nodes 218B can transmit the output of the one or more calculations to the output nodes 218C. In some embodiments, the output nodes 218C can generate output data of the neural network 218. The nodes of different layers can be connected via connectors 220. In some embodiments, the layers can be fully connected. In some embodiments, the layers may not be fully connected.

In some embodiments, a number of input nodes 218A in the neural network 218 can be based on a number of dimensions of input data and a number of inputs. For example, if the input data includes a one-dimensional array of N inputs, the neural network 218 may comprise N input nodes 218A. In some embodiments, a number of output nodes 218C in the neural network can be based on a number of dimensions of the output data, a number of outputs, etc. For example, if the output data includes a two-dimensional array of N×M outputs, the neural network 218 may comprise N×M output nodes 218C.

In some embodiments, the number of input nodes 218A and/or the number of output nodes 218C in the neural network 218 can be based on a number of desired data sources 104 and/or outputs 116. For example, if the neural network 218 receives data from two data sources 104, the neural network 218 may comprise two concatenated groups of input nodes 218A.

In some embodiments, a user can determine the number of input nodes 218A and/or the number of output nodes 218C. In some embodiments, the encoder 206 may comprise one or more concatenated groups of input nodes 218A, etc. In some embodiments, the fusor 210 may comprise one or more concatenated groups of input nodes 218A. In some embodiments, the encoder 206 may comprise one or more concatenated groups of input node 218A.

In some embodiments, the user can determine a structure of the neural network 218. In some embodiments, the user can determine the number of hidden nodes 218B in the neural network 218, the number of layers of hidden nodes 218B, or both. In some embodiments, the user can determine one or more connections 220 between each hidden node 218B (e.g., between each layer of hidden nodes). In some embodiments, the specific configuration of a neural network can be determined automatically.

In some embodiments, the neural network 218 may comprise a mathematical model. In some embodiments, the user can select one or more portions of the mathematical model. For example, different portions of the neural network 218 may be used when providing different input data to the neural network, when using the neural network 218 to produce different outputs, and so forth. In some embodiments, a user may not select particular portions to use. Instead, different portions of the neural network can be used during deployment as a result of training a model to perform a particular task or to work with particular types of input data.

In some embodiments, the input nodes 218A, hidden nodes 218B, output nodes 218C, or any combination thereof may comprise a weight. In some embodiments, the weights of each input node 218A, hidden node 218B, and/or output node 218C can be modified to modify the output data of the neural network.

In some embodiments, the encoders 206, the fusor 210, the decoders 214, or any combination thereof may comprise separate AI/ML models. In some embodiments, the encoders 206, the fusor 210, and/or the decoders 214 may comprise portions of the model 200. In some embodiments, the user can select one or more of the encoders 206, the fusor 210 and/or the decoders 214 based on a task or function. In some embodiments, the model 200 may comprise only the selected one or more of the encoders 206, the fusor 210 and/or the decoders 214.

### AI/ML Model Training

FIG. 3A shows a schematic of a system 300 for training the model 200, an encoder 206, a fusor 210, and/or a decoder 214. In some embodiments, the system 300 may comprise data sources 104, an encoder 206 for each data source 104, a fusor 210, outputs 116, and a decoder 214 for each output. In some embodiments, the encoders 206, the fusor 210, and the decoders 214 can be trained at a same time.

FIG. 3B shows a flowchart of a process 350 for training the model 200, the encoder 206, the fusor 210, the decoder 214, or any combination thereof. In some embodiments, at operation 352, a user can configure the encoders 206, the fusor 210, and/or the decoders 214, for example by setting one or more initial weights. In some embodiments, the user can configure the structure of the neural network 218 of the encoders 206, the fusor 210, and/or the decoder 214. In some embodiments, the user can determine or select a number of input nodes 218A and/or output nodes 218C of the encoders 206, the fusor 210, and/or the decoders 214. In some embodiments, the user can determine or select one or more portions of the mathematical model of the neural network 218 of the encoders 206, the fusor 210, and/or the decoders 214.

In some embodiments, at operation 354, the system 300 can input the input data from the data sources 104 into the corresponding encoders 206. In some embodiments, the encoders 206 can each generate a latent vector 208.

In some embodiments, at operation 356, the system 300 and/or the encoders 206 can transmit the latent vectors 208 to the fusor 210. In some embodiments, the fusor 210 can receive the latent vectors 208. In some embodiments, the fusor 210 can fuse, merge, unify, join, or otherwise combine the latent vectors 208 to generate a fused latent vector 212.

In some embodiments, at operation 357, the system 300 and/or the fusor 210 can transmit the fused latent vector 212 to the decoders 214. In some embodiments, each decoder 214 can receive the fused latent vector 212. In some embodiments, each decoder 214 can generate an output 116.

In some embodiments, at operation 358, the system 300 can compare the output 116 of a decoder to a desired output for each decoder 214. In some embodiments, the desired output may comprise a reconstruction of the input data, a diagnosis, an identification of one or more features in the input data, a recommendation for one or more tests to perform, a prognosis determination, and/or any other clinical determination or decision.

In some embodiments, the system 300 can automatically compare the output 116 to the desired output to determine an error between the output 116 and the desired output. In some embodiments, if the desired output is a reconstruction of the input data or identification of one or more features in the input data, the error may comprise a percentage or other numerical representation of the differences and/or the similarities between the output 116 and the desired output. In some embodiments, if the desired output is a diagnosis, identification of one or more features in the input data, a recommendation for one or more tests to perform, a prognosis determination, and/or any other clinical determination or decision, the error may comprise or indicate whether the output 116 is the same as the desired output or the same as the desired output within a defined threshold. In some embodiments, when model is used for classification problems, the error can be based on how similar the output classification is to the true classification. As an example, a model configured for diagnosis can have a smaller error if the output diagnosis is in a same disease category as the true diagnosis than if the output diagnosis is in a different disease category than the true diagnosis.

At operation 359, the system can determine if the error is below a threshold amount. If so, the process 350 can stop. If not, the process can continue to operation 360.In some embodiments, based on the error determined by the system 300 at operation 358, the system 300 can automatically modify or adjust the encoders 206, the fusor 210, the decoders 214, or any combination thereof at operation 360. In some embodiments, the system 300 can modify or adjust the neural network 218 of the encoders 206, the fusor 210, the decoders 214, or any combination thereof. In some embodiments, the system 300 can modify or adjust the weights of the input nodes 218A, the hidden nodes 218B, the output nodes 218C, or any combination thereof. In some embodiments, the system 300 can modify or adjust the encoders 206, the fusor 210, the decoders 214, or any combination thereof to reduce, minimize, or eliminate the error.

In some embodiments, after the system 300 modifies or adjusts the encoders 206, the fusor 210, the decoders 214, or any combination thereof, the system 300 can repeat operations 354-360. In some embodiments, the system 300 can repeat operations 354-360 until the error determined at operation 358 is zero. In some embodiments, the system 300 can continue to repeat operations 354-360 until the error determined at operation 358 is below a predetermined threshold.

In some embodiments, the system 300 can repeat operations 354-360 for a fixed number of iterations, up to a maximum number of iterations, or until an error is below a predetermined threshold.

In some embodiments, each time the system 300 repeats operations 354-360, the encoders 206 can automatically determine which information to include in the latent vectors 208. The encoders 206 can determine which information to include in the latent vectors 208 based on the error determined at operation 360 for each output 116. In some embodiments, as the system 300 reduces, minimizes, or eliminates the error, the encoders 206 can automatically learn or determine the information to include in the latent vectors 208 that results in the reduced, minimized, or eliminated error.

In some embodiments, each time the system 300 repeats operation 354-360, the fusor 210 can automatically determine which information to include in the fused latent vector 212. The fusor 210 can determine which information to include in the fused latent vector 212 based on the error determined at operation 360 for each output 116. In some embodiments, as the system 300 reduces, minimizes, or eliminates the error, the fusor 210 can automatically learn or determine the information to include in the latent vectors 208 that results in the reduced, minimized, or eliminated error for each output 116. In some embodiments, the information in the fused latent vectors 212 that results in the reduced, minimized, or eliminated error for each output 116 may comprise the relevant information.

In some embodiments, each time the system 300 repeats operations 354-360, the decoders 214 can automatically determine which information from the fused latent vector 212 the decoders 214 can use to generate the output 116. In some embodiments, the decoders 214 can determine which information from the fused latent vector 212 the decoders 214 can use to generate the output 116 based on the error determined at operation 360 for each output 116. In some embodiments, as the system 300 reduces, minimizes, or eliminates the error, the decoders 214 can automatically learn or determine which information from the fused latent vector 212 the decoders 214 can use to generate the output 116 that results in the reduced, minimized, or eliminated error.

It is to be appreciated that although the system 300 is shown with two encoders 206, one fusor 210, and two decoders 214, in some embodiments, the system 300 may comprise any number of encoders 206, fusors 210 and/or decoders 214. In some embodiments, the number of encoders 206 can be based on a number of data sources 104. In some embodiments, the number of decoders 214 can be based on a number of different outputs 116 the system 300 can generate. In some embodiments, the system 300 may comprise a same number of encoder 206 and decoders 214. In some embodiments, the system 300 may comprise a different number of encoders 206 and decoders 214.

FIG. 4A illustrates an approach for training an encoder and/or fusor according to some embodiments. In some embodiments, a system can create a latent vector 208A using an encoder 206A and data from data source 104A. The system can generate a latent vector 208B from data in a data source 104B using a second encoder 206B. In some embodiments, the first encoder 206A and the second encoder 206B can be the same encoder or can be different encoders. In some embodiments, the system can fuse the first latent vector 208A and the second latent vector 208B using a first fusor 210A, which can output a fused latent vector 212A. In some embodiments. The system can fuse the first fused latent vector 212A and the second latent vector 208B using a second fusor 210B. The second fusor 210B can be the same fusor as the first fusor 210A. The second fusor 210B can output a second fused latent vector 212B. The system can compare the second fused latent vector 212B and the first fused latent vector 212A to determine a difference between the second fused latent vector 212B and the first fused latent vector 212A. Since the second latent vector 208B was fused to the first fused latent vector 212A, which already included information from the second latent vector 208B, it can be expected that the fused latent vector 212B is the same or nearly the same as the first fused latent vector 212A. In some embodiments, one or more weights of the first fusor 210A can be adjusted until the second fused latent vector 212B matches the first fused latent vector 212A, such that redundant information is not added to the first fused latent vector 212A when it is fused with second latent vector 208B.

FIG. 4B shows a schematic of a method 450 for training a fusor to automatically determine redundant information. In some embodiments, at operation 452, the system 400 can input or transmit input data from the data sources to the encoders to generate latent vectors. In some embodiments, the encoders can receive the input data from the data sources. In some embodiments, the encoders can each generate a latent vector from the received input data. In some embodiments, each encoder can be configured to generate a latent vector from a particular data source.

In some embodiments, at operation 454, the fusor can receive the latent vectors 208. For example, in some embodiments, the system 400 or the encoders 206 can transmit the latent vectors 208 to the fusor 210. In some embodiments, the fusor can fuse, merge, unify, join, or otherwise combine the latent vectors to generate a first fused latent vector.

In some embodiments, at operation 456, the fusor can receive the first fused latent vector and at least one latent vector. For example, in some embodiments, at operation 456, the system can provide the first fused latent vector and at least one latent vector to the fusor. In some embodiments, the fusor can fuse, merge, unify, join, or otherwise combine the first fused latent vector and the at least one latent vector to generate a second fused latent vector.

In some embodiments, at operation 458, the system can compare the first fused latent vector and the second fused latent vector. In some embodiments, the system can automatically determine information in the second fused latent vector that is included in the first fused latent vector. In some embodiments, redundant information may comprise the information in the second fused latent vector that is included in the first fused latent vector. In some embodiments, the fusor can receive an indication of which information is redundant information. In some embodiments, the fusor can use the indication of which information is redundant information to automatically learn which information is redundant information.

In some embodiments, based on the indication of which information is redundant information, the system can automatically modify or adjust a neural network of the fusor at operation 460. In some embodiments, the system can modify weights of the input nodes, the hidden nodes, and/or the output nodes. In some embodiments, the system can modify or adjust the fusor to automatically determine that the information in the second fused latent vector that is included in the first fused latent vector is redundant information.

In some embodiments, the system can repeat method 450 one time or a plurality of times with a plurality of input data from a plurality of data sources. In some embodiments, the system can repeat method 450 until the fusor can automatically identify redundant information so the fusor does not include redundant information in a fused latent vector. In some embodiments, the system can repeat method 450 until the fusor does not include information from a first latent vector that is already in a fused latent vector and/or a second latent vector.

### Latent Vector Completeness

In some embodiments, multiple latent vectors can be fused with one or more other latent vectors, with one or more fused latent vectors, etc. There can be limited usefulness to additional fusing operations, as the change in information represented by a fused latent vector may stop improving or may improve by less than a threshold amount.

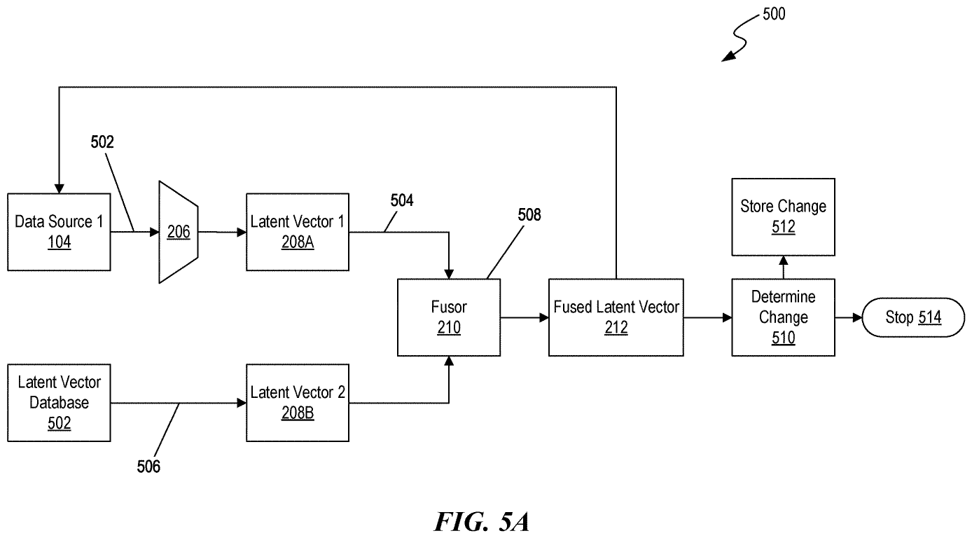

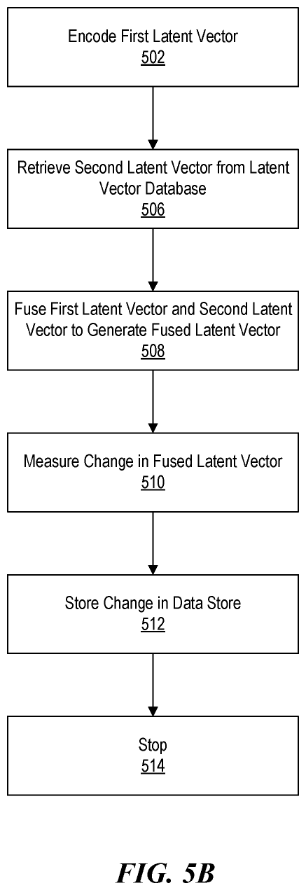

FIG. 5A shows a schematic of a method 500 for determining completeness of a latent vector. In some embodiments, at operation 502, an encoder 206 can receive input data from a data source 104. In some embodiments, the encoder 206 can generate a first latent vector 208A.

In some embodiments, at operation 504, the encoder 206 can transmit the first latent vector 208A to a fusor 210. In some embodiments, the fusor 210 can receive the first latent vector 208A from the encoder 206. In some embodiments, the fusor 210 may comprise a trained fusor 210. In some embodiments, the fusor 210 can be trained to determine redundant information via system 400 and method 450.

In some embodiments, at operation 506, the fusor 210 can retrieve a second latent vector 208B from a latent vector database. In some embodiments, the first latent vector 208A and the second latent vector 208B may comprise latent vectors generated from input data captured at different times. In some embodiments, the first latent vector 208A and the second latent vector 208B may comprise latent vectors generated from input data captured at a same time. In some embodiments, the first latent vector 208A and the second latent vector 208B may comprise latent vectors generated from input data captured from a same data source or same modality. In some embodiments, the first latent vector 208A and the second latent vector 208B may comprise latent vectors generated from input data captured from different data sources or modalities.

In some embodiments, at operation 508, the fusor 210 can generate a fused latent vector 212. In some embodiments, the fused latent vector 212 may comprise the information in the first latent vector 208A and the second latent vector 208B. In some embodiments, the fused latent vector 212 may comprise all of the information in the first latent vector 208A and the information in the second latent vector 208B that is not redundant information. In some embodiments, the fused latent vector 212 may comprise all of the information in the second latent vector 208B and the information in the first latent vector 208A that is not redundant information.

In some embodiments, at operation 510, model 200 and/or the fusor 210 can determine a change between the second latent vector 208B and the fused latent vector 212. In some embodiments, at operation 510, the fusor 210 can compare the change between the second latent vector 208B and the fused latent vector 212 to a predetermined change threshold. In some embodiments, the change and/or the predetermined change threshold may comprise a percentage or other numerical representation of an amount of different data between the second latent vector 208B and the fused latent vector 212. In some embodiments, the change and/or the predetermined change threshold may comprise a percentage or other numerical representation of an amount of information between the second latent vector 208B and the fused latent vector 212 that is the same information. In some embodiments, the predetermined change threshold can be based on a function of the model 200. For example, in some embodiments, if the model 200 is used for diagnosis, the predetermined change threshold may comprise a first threshold, and if the model 200 is used for surgery or other treatment, the predetermined change threshold may comprise a second threshold. For example, a larger change threshold may be used during diagnosis, where additional information may be of lesser importance, and a smaller change threshold can be used during surgical or other treatment, when even small changes in information may significantly impact planning, treatment, and/or outcomes.

In some embodiments, at operation 512, the model 200 can store the fused latent vector 212 in the latent vector database and store the determined change.

In some embodiments, if the determined change is below the predetermined change threshold, the model 200 can determine that the fused latent vector 212 is complete, and at operation 514, the model 200 and/or the fusor 210 can stop fusing latent vectors into the fused latent vector 212.

In some embodiments, if the determined change is above a predetermined change threshold, the model 200 can determine that the fused latent vector 212 is incomplete, and the model 200 can repeat operations 502-512 of method 500. In some embodiments, the model 200 can repeat operations 502-512 of method 500 until the determined change is below the predetermined change 512 of method 500 until the determined change is below the predetermined change threshold. In some embodiments, the model 200 can repeat operations 502-512 with input data from a same data source 104 each time the model 200 repeats operations 502-512. In some embodiments, the model 200 can repeat operations 502-512 with input data from a different data source 104 each time the model 200 repeats operations 502-512. In some embodiments, when the AI/ML model repeats operations 502-512 of method 500, the second latent vector 208B may comprise the fused latent vector 212.

In some embodiments, if the determined change is above the predetermined change threshold, the model 200, the fusor 210, and/or a decoder 214 can determine that more input data or information is required to complete the fused latent vector 212. In some embodiments, if the data source 104 does not include any new input data to repeat operation 502-512, the model 200, the fusor 210, and/or a decoder 214 can determine a different data source 104 that can provide enough input data to add to the fused latent vector 212 so the determined change is above the predetermined change threshold.

In some embodiments, the model 200, the fusor 210, and/or the decoder 214 can analyze a plurality of other fused latent vectors 212 to determine which different data source 104 can provide enough input data. In some embodiments, the model 200, the fusor 210, and/or the decoder 214 can analyze the plurality of other fused latent vectors 212 that were generated from input data provided from the same data source 104. In some embodiments, the model 200, the fusor 210, and/or the decoder 214 can determine which different data source 104 provided enough input data to add to the fused latent vector 212 so the determined change is above the predetermined change threshold. For example, in some embodiments, if the data source 104 is an SLO imaging device, and a plurality of other fused latent vectors 212 generated from input data from the SLO imaging device were previously determined to be incomplete by the model 200, the fusor 210, and/or the decoder 214, and an OCT imaging device provided enough input data to add to the fused latent vector 212 so the determined change was above the predetermined change threshold, the model 200, the fusor 210, and/or the decoder 214 can determine that fusing a latent vector 208 that includes input data from an OCT imaging device into the fused latent vector 212 will modify the fused latent vector 212 so the determined change is above the predetermined change threshold.

In some embodiments, the model 200, the fusor 210, and/or the decoder 214 can analyze the plurality of other fused latent vectors 212 to predict what the determined change will be if input data from a different data source 104 is added or included in the fused latent vector 212. In some embodiments, the model 200, the fusor 210, and/or the decoder 214 can generate one or more recommendations of which different data source 104 from which to provide input data. In some embodiments, based on the one or more recommendations, a user can capture data with the different data source 104.

FIG. 5B is a flowchart that illustrates a process for measuring change in a fused latent vector according to some embodiments. At operation 502, a system can encode a first latent vector. At operation 506, the system can retrieve a second latent vector from a latent vector database. The second latent vector can, in some embodiments, be a latent vector captured at an earlier time. At operation 508, the system can fuse the first latent vector and the second latent vector to generate a fused latent vector. At operation 510, the system can determine a change in the fused latent vector, for example by comparing the fused latent vector to a previous latent vector. In some embodiments, the system can store the change in a data store at operation 512. At operation 514, the process can stop. In some embodiments, operations 502 through 512 can be repeated.

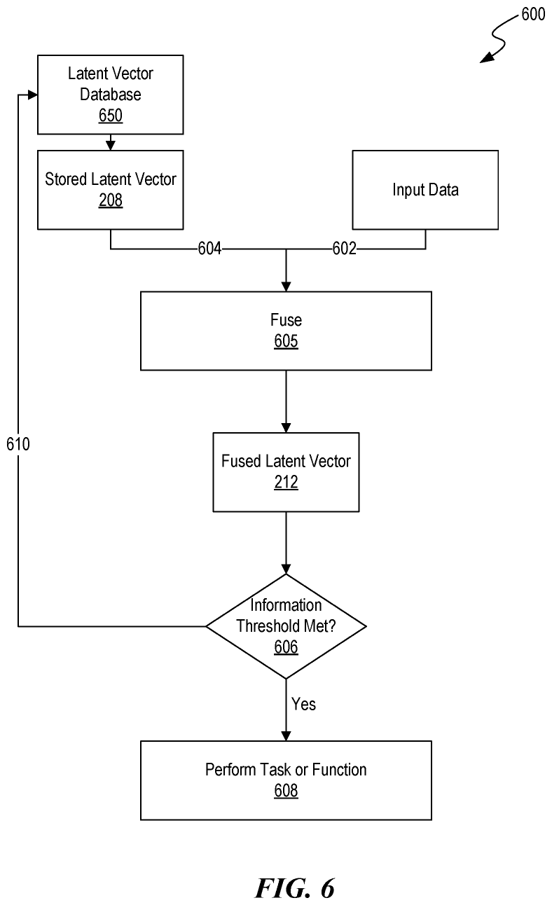

FIG. 6 shows a schematic of an approach for determining latent vector completeness according to some embodiments. The approach illustrated in FIG. 6 can be carried out on a computer system or multiple computer systems (generally “system”). In some embodiments, the method illustrated in FIG. 6 can be implemented using real time or substantially real time input data, although this may not be the case in other embodiments. In some embodiments, at operation 602, a data source can capture input data. In some embodiments, the data source can transmit the input data to the model in real time or substantially real time. In some embodiments, the data source can transmit the input data to an encoder. In some embodiments, the encoder can generate a latent vector from the input data.

In some embodiments, at operation 604, the system can retrieve a stored latent vector 208, for example from a latent vector database 650.

In some embodiments, at operation 605, a fusor can fuse, merge, unify, join, or otherwise combine the latent vector generated by the encoder from the input data and the latent vector retrieved from the latent vector database to generate a fused latent vector 212.

In some embodiments, at operation 606, the system can determine if an amount of information included in the fused latent vector is above an information threshold. In some embodiments, the information threshold may comprise a percentage of a total amount of information the fused latent vector can store. In some embodiments, the information threshold may comprise an amount of information, for example, a number of bits of information. In some embodiments, the information threshold may comprise a predetermined change threshold or completeness threshold as described with reference to FIGS. 5A and 5B.

In some embodiments, the information threshold can be based on a task or function the fused latent vector will be used for. For example, in some embodiments, the task or function may comprise treatment, such as floater removal or breaking up floaters, detection of one or more conditions, tracking an indication of one or more conditions, predicting a likelihood of a patient having one or more conditions in the future, predicting an outcome of a surgery, and/or any other clinical task or function. In some embodiments, if the task or function the fused latent vector will be used for includes surgery, the information threshold may be greater or larger than the information threshold for detection of one or more conditions.

In some embodiments, a system can determine the amount of information included in the fused latent vector and compare said amount of information included in the fused latent vector to the information threshold. In some embodiments, the amount of information included in the fused latent vector may comprise the percentage of the total amount of information the fused latent vector can store that is included in the fused latent vector. In some embodiments, the model and/or the fusor can compare the percentage of the total amount of information the fused latent vector can store to the information threshold. In some embodiments, the amount of information included in the fused latent vector can be a change between the latent vector retrieved from the latent vector database and the fused latent vector generated by the fusor as described with reference to FIGS. 5A and 5B. In some embodiments, the system can compare the determined change to the information threshold.

In some embodiments, if, at operation 606, the system determines that the amount of information included in the fused latent vector is greater than the information threshold, the system can, at operation 608, the system can perform the task or function, such as applying a decoder to the fused latent vector.

In some embodiments, if, at operation 606, the system determines the amount of information included in the fused latent vector is less than or below the information threshold, at operation 610, the system can store the fused latent vector in the latent vector database 650. In some embodiments, the model can repeat operations 602-606 of method 600 until the model and/or the fusor determines the amount of information included in the fused latent vector is greater than the information threshold. In some embodiments, each time the system repeats operations 602-606, the latent vector retrieved at operation 604 may comprise the fused latent vector from a previous time the system performed operations 602-606.

### Latent Vector Applications

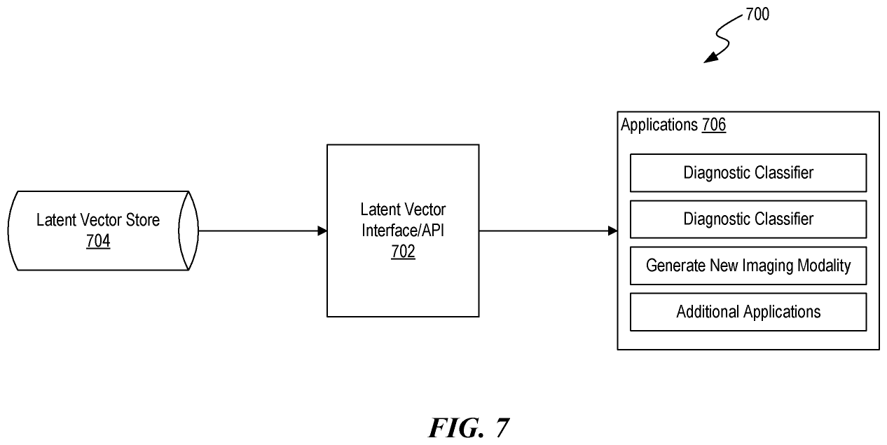

FIG. 7 shows a schematic of a system 700 for latent vector applications. In some embodiments, the system 700 may comprise a latent vector interface 702. In some embodiments, the latent vector database 704 may comprise a plurality of latent vectors, fused latent vectors, or both. In some embodiments, the latent vector database 704 may comprise a database of trained encoder and fusor models. In some embodiments, the latent vector database 704 may comprise a plurality of latent vectors, fused latent vectors, or both of a plurality of patients. In some embodiments, each of the plurality of patients may have a latent vector, a fused latent vector, or both for each condition, organ, portion of the patient's body, treatments plan, surgery, etc., associated therewith.

In some embodiments, the latent vector interface 702 may comprise an application programming interface (API). In some embodiments, a user, such as a clinician, can access a latent vector database 704 via the latent vector interface 702 (e.g., via an application, web page, etc., configured to utilize the latent vector interface 702). In some embodiments, the user can access the latent vector database 704 via a user device, such as a personal computer, cellular phone, smartphone, laptop, tablet computer, and/or any other computing device.

In some embodiments, system 700 may comprise a plurality of applications 706. In some embodiments, the user device may comprise the plurality of applications 706 (e.g., locally installed applications on a user device). In some embodiments, the plurality of applications 706 can be stored in an application database or an application platform. In some embodiments, the application database or application platform may comprise one or more third-party applications.

In some embodiments, each application 706 may comprise at least a portion of a model as described herein. In some embodiments, each application 706 may comprise one or more encoders, one or more fusors, and/or one or more decoders. In some embodiments, each application 706 can be configured to generate an output and/or perform a task or function. For example, an application 706 can be configured to aid in diagnosing a patient with one or more conditions, generate an image from a imaging device or modality that was not used to generate the latent vector and/or fused latent vector (e.g., to generate an SLO image from OCT images, or to generate a CT scan based on MRI data), verify a patient's identity, predict a likelihood of the patient having one or more conditions in the future, predict an outcome of a surgery, predict development of one or more conditions over time, perform a surgery, generate instructions for an imaging and/or surgical device, recommend treatment options with projected outcomes, and/or any other clinical task or function.

In some embodiments, an application 706 can be configured to function with particular latent vectors, fused latent vectors, or both. In some embodiments, the application 706 can be configured to perform a task or function for latent vectors, fused latent vectors, or both that include information about a particular organ, location of the patient's body, and/or particular condition.

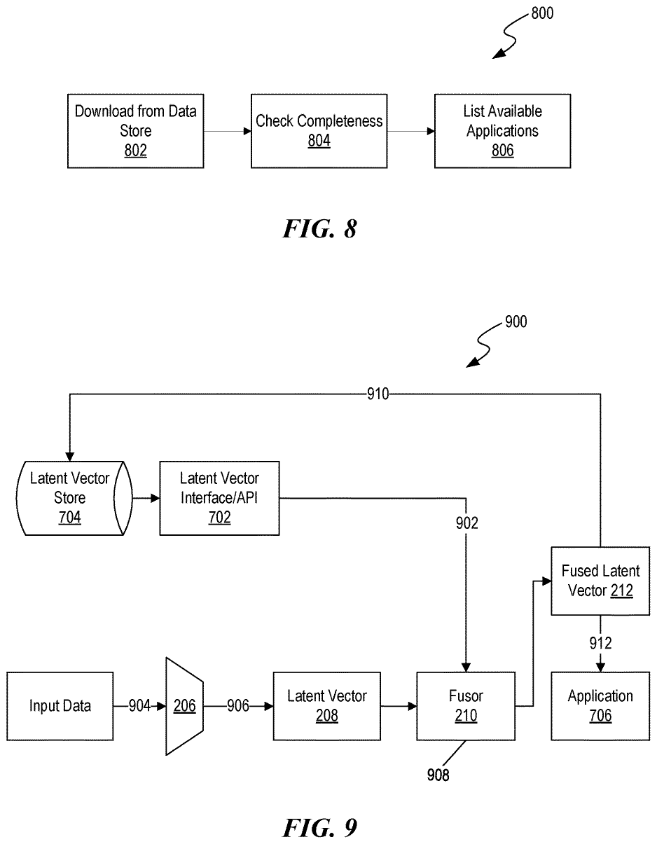

FIG. 8 shows a schematic of a method 800 for determining available applications. As described herein, applications for which a latent vector (or fused latent vector) can vary depending on the content of the latent vector or fused latent vector, amount of information in the latent vector or fused latent vector, etc. In some embodiments, at operation 802, a system can access a latent vector from a data store, for example via an API.

In some embodiments, at operation 804, the system can determine completeness of the one or more latent vectors and/or fused latent vectors via, for example via the approaches described with respect to FIGS. 5A and 5B. In some embodiments, the system can determine completeness of the one or more latent vectors and/or fused latent vectors. In some embodiments, when a system transmits one or more latent vectors and/or fused latent vectors, a receiving user device can download or retrieve at least a portion of a model. In some embodiments, the portion of the model may comprise a portion of the model configured to perform method 500. In some embodiments, at least a portion of the model can be stored on the user device. In some embodiments, no part of the model may be stored on the user device. Instead, in some embodiments, a model can be hosted on a cloud platform and accessed via a network.

In some embodiments, at operation 806, the system can determine and/or display to the user a list of available applications. In some embodiments, the list of available applications may comprise one or more applications configured to function with the one or more latent vectors and/or fused latent vectors. In some embodiments, the system allows uploading, storage, and retrieval of AI/ML models. In some embodiments, the available applications can depend on, for example, the completeness of the latent vector or fused latent vector.

FIG. 9 shows a schematic of a method 900 for including additional information in a latent vector and/or a fused latent vector. In some embodiments, at operation 902, a system can retrieve or download a latent vector and/or a fused latent vector from the latent vector database 704, for example using the API 702, although in some embodiments the API 702 may not be used.

In some embodiments, at operation 904, a system can receive input data and pass the input data to an encoder 206. In some embodiments, the input data can be captured by a data source in real time or substantially real time. In some embodiments, a user can store the input data on a user device and transmit the input data from the user device to the system. In some embodiments, the user device and the system can be a same system.

In some embodiments, at operation 906, the encoder 206 can generate a latent vector 208 from the input data. In some embodiments, the system can transmit the latent vector 208 generated from the input data to the fusor 210.

In some embodiments, at operation 908, the fusor 210 can fuse, merge, unify, join, or otherwise combine the latent vector 208 generated from the input data with the latent vector or fused latent vector from the latent vector database 704. In some embodiments, the fusor can generate a fused latent vector 212 that may comprise the latent vector 208 generated from the input data or may comprise a representation of the latent vector 208.

In some embodiments, at operation 910, the system can store the fused latent vector 212 in the latent vector database 704. In some embodiments, the fused latent vector 212 may replace the latent vector or fused latent vector that was retrieved from the latent vector database 704. In some embodiments, the fused latent vector 212 may be stored along with the latent vector or fused latent vector was retrieved from the latent vector database 704.

In some embodiments, at operation 912, the system 700, the system provides the fused latent vector 212 to an application 706.

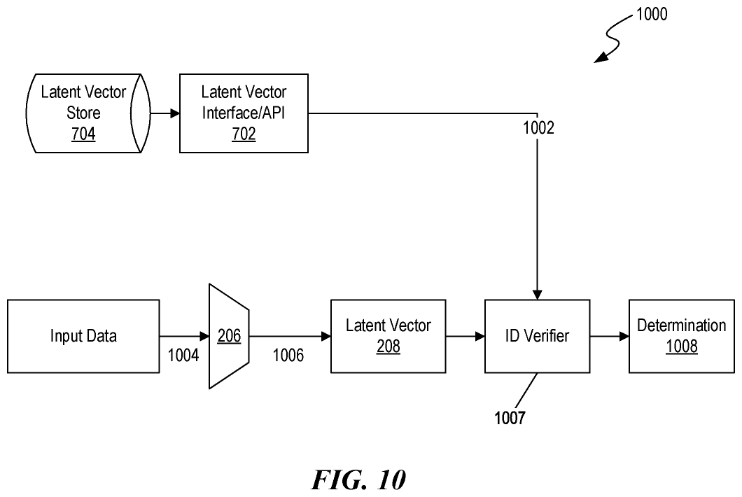

FIG. 10 shows a schematic of a method 1000 for verifying a patient's identity according to some embodiments. In some embodiments, at operation 1002, a system can retrieve a latent vector or fused latent vector from a latent vector database 704. In some embodiments, the system can retrieve the latent vector or fused latent vector from the latent vector database 704 via an API 702, although the API may not be used in some other embodiments. At operation 1004, the system can receive input data and can pass the input data to an encoder 206. At operation 10006, the encoder 206 can generate a latent vector 208. At operation 1007, an identity verifier can verify the identity of the patient based on the latent vector 208 and the latent vector or fused latent vector retrieve from the latent vector database 704. For example, in some embodiments, the latent vector 208 can be fused with the latent vector or fused latent vector retrieved from the latent vector database 704 and a different between the new fused latent vector and the latent vector or fused latent vector retrieved from the latent vector database 704 can be determined by the system. In some embodiments, the system can compute a similarity (e.g., cosine similarity) between the latent vector 208 and the latent vector or fused latent vector retrieved from the latent vector database 704. In some embodiments, the latent vector or fused latent vector in the latent vector database 704 can be associated with a specific patient or individual. In some embodiments, the individual's identity can be considered as verified if the similarity between latent vector 208 and the latent vector or fused latent vector retrieved from the latent vector database 704 is above a threshold amount.

In some embodiments, the input data can be captured in real time or substantially real time, for example to enable real time or nearly real time identity verification (e.g., fingerprint verification, facial verification, iris verification, etc.). In some embodiments, a user can store the input data on a user device and transmit the input data from the user device to the system.

In some embodiments, at operation 1006, the encoder 206 can generate a latent vector 208 from the input data. In some embodiments, the encoder 206 can be trained as described above with reference to FIGS. 3A and 3B. In some embodiments, the encoder 206 can be trained to generate a latent vector 208 specifically for verifying a patient's identity.

The identity verifier can output a determination 1008. In some embodiments, the determination can be binary (e.g., verified or not verified). In some embodiments, the determination can be a confidence score.

The process 1000 can be used in various applications. For example, the process 1000 can be used for identity verification for access to a building, campus, etc., to enable functionality on machinery, computers, etc., or to verify a patient's identity. For example, in some embodiments, the process 1000 can be used to verify identity prior to performing a surgical procedure on a patient.

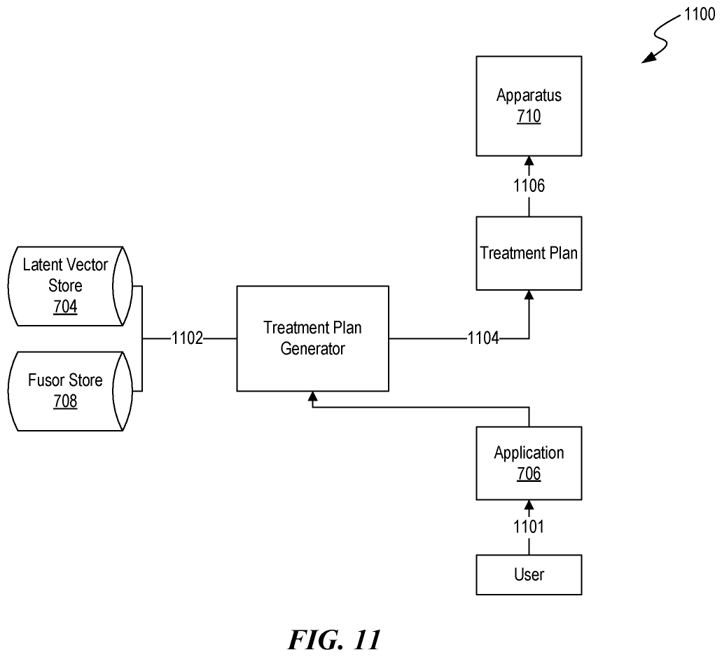

FIG. 11 shows a schematic of a method 1100 for generating a treatment plan according to some embodiments. In some embodiments, at operation 1101, a user can select and interact with an application 706 configured to generate a treatment plan.

In some embodiments, at operation 1102, the application 706 can be configured to interact with a treatment plan generator to generate a treatment plan. The treatment plan generator can retrieve one or more latent vectors and/or fused latent vectors from the latent vector database 704. The treatment plan generator can retrieve or select one or more fusors from a fusor database 708. In some embodiments, the fusor database 708 may comprise one or more fusors. In some embodiments, the fusor database 708 may comprise one or more encoders and/or one or more decoders, which can be used by the treatment plan generator to generate a treatment plan.

In some embodiments, the one or more fusors, the one or more encoders, and/or the one or more decoders may comprise organ-specific and/or target-specific fusors, encoders and/or decoders. In some embodiments, a treatment plan is retrieved from a database based on a condition diagnosed, state of a target, threshold for intervention, etc.

In some embodiments, at operation 1104, the application 706 configured to generate a treatment plan can generate a treatment plan. In some embodiments, the treatment plan generator can generate a treatment plan using the retrieve vectors, latent vectors, fusors, encoders, and/or decoders.

In some embodiments, at operation 1106, system can transmit the treatment plan to a treatment apparatus 710. In some embodiments, the treatment apparatus 710 may comprise an apparatus or device configured to administer treatment to the user. In some embodiments, the treatment apparatus 710 may comprise a surgical device or system, and/or any other clinical treatment device. In some embodiments, the apparatus 710 can comprise a laser, for example a YAG laser which can be used, for example, for treating floaters.

### Treatment

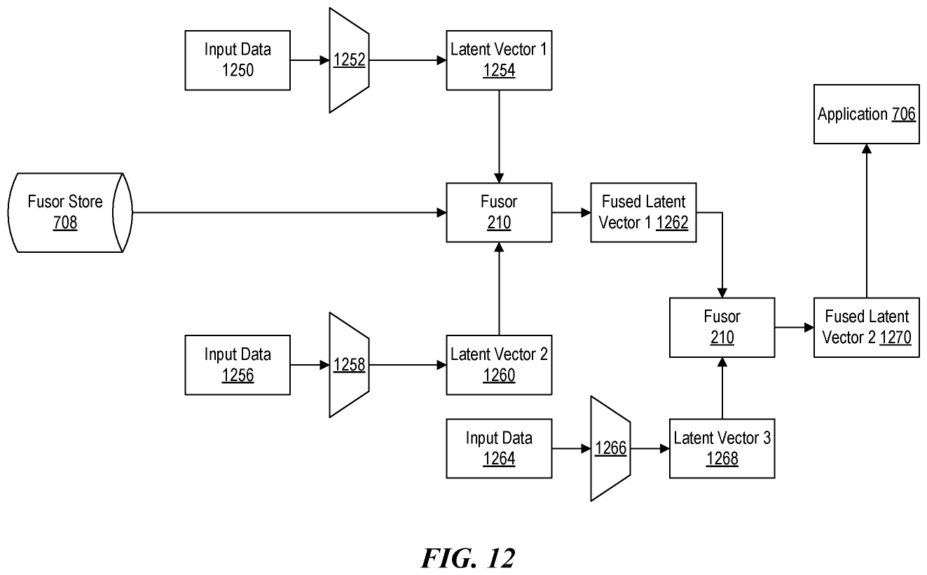

In some embodiments, the approaches described herein can be used in real time or substantially real time during treatment. FIG. 12 shows a method and components of a system that can be used for performing a treatment procedure utilizing the approaches described herein.

In some embodiments, a system can access a fusor 210. In some embodiments, the fusor 210 can be accessed from a fusor store 708, although in some other embodiments the fusor store 708 may not be involved. The system can receive input data 1250, which can be encoded by an encoder 1252 to generate a first latent vector 1254. The system can receive input data 1256, which can be provided to an encoder 1258 to produce a second latent vector 1260. The fusor 210 can receive the first latent vector 1254 and the second latent vector 1260 and generate a first fused latent vector 1262. In some embodiments, the first fused latent vector 1262 can include relevant information from the first latent vector 1254 and the second latent vector 1260. In some embodiments, the relevant information can include, for example, relevant information as described herein with reference to FIG. 2A. A third encoder 1266 can receive input data 1264 and code a third latent vector 1268. The fusor 210 can receive the third latent vector 1268 and the first fused latent vector 1262 and can generate a second fused latent vector 1270. The system can provide the second fused latent vector 1270 to an application 706.

In some embodiments, the fusor can be run on a server, cluster, etc. In some embodiments, the fusor can be run on a user device, such as a desktop, tablet, smartphone, etc. In some embodiments, running locally on a user a user device can offer improved performance over running remotely and/or can offer improved privacy or reduced information sharing concerns.

In some embodiments, the input data 1250 can be captured or received at a first time, for example at the start of a surgical procedure or other treatment. In some embodiments, the first time may comprise a start of the treatment. In some embodiments, the first time may comprise a time before the start of the treatment, for example, a visit with the clinician before a day of the treatment, the day of the treatment when the patient is being prepped for treatment, when the patient is in the operating room or procedure room before the start of treatment, etc. In some embodiments, the first time may comprise any time throughout the treatment.

In some embodiments, the first latent vector 1254 can be produced as soon as (or soon after) the input data 1250 is available. In some embodiments, the first latent vector 1254 may not be generated until there is a need for the first latent vector 1254 or until additional data (e.g., input data 1256) is received.

In some embodiments, the input data 1256 can be captured at a second time. In some embodiments, the input data 1256 can be received from a same data source (e.g., a same imaging device) as the input data 1250. In some embodiments, the input data 1256 can be received from a different data source than the input data 1250. In some embodiments, the second time may comprise a time after the first time. In some embodiments, the second time can be shortly after the first time, for example after less than 1 ms, less than 10 ms, less than 100 ms, or less than 1 s. In some embodiments, the second time can be a longer time after the first time, for example minutes, hours, days, weeks, or months. In some embodiments, the second time can be the same or substantially the same as the first time.

In some embodiments, the input data 1264 can be captured at a third time. In some embodiments, the input data 1264 can be captured by a same data source or a different data source than the input data 1256, the second latent vector 1260, or both. In some embodiments, the third time may comprise a time after the second time. In some embodiments, a time between the third time and the second time may comprise a same time as a time between the first time and the second time. In some embodiments, the time between the third time and the second time may comprise a different time from the time between the first time and the second time. In some embodiments, the third time may comprise a time of about 0.1 ms, about 0.5 ms, about 1 ms, about 2 ms, about 3 ms, about 4 ms, about 5 ms, about 6 ms, about 7 ms, about 8 ms, about 9 ms, about 10 ms, about 11 ms, about 12 ms, about 13 ms, about 14 ms, about 15 ms, about 16 ms, about 17 ms, about 18 ms, about 19 ms, about 20 ms, about 21 ms, about 22 ms, about 23 ms, about 24 ms, about 25 ms, about 26 ms, about 27 ms, about 28 ms, about 29 ms, about 30 ms, about 31 ms, about 32 ms, about 33 ms, about 34 ms, about 35 ms, about 36 ms, about 37 ms, about 38 ms, about 39 ms, about 40 ms, about 41 ms, about 42 ms, about 43 ms, about 44 ms, about 45 ms, about 46 ms, about 47 ms, about 48 ms, about 49 ms, about 50 ms, about 60 ms, about 70 ms, about 80 ms, about 90 ms, about 100 ms, about 150 ms, about 200 ms, about 250 ms, about 300 ms, about 350 ms, about 400 ms, about 450 ms, about 500 ms, about 550 ms, about 600 ms, about 650 ms, about 700 ms, about 750 ms, about 800 ms, about 850 ms, about 900 ms, about 950 ms, about 1 second, about 2 seconds, about 3 seconds, about 4 seconds, about 5 seconds, about 6 seconds, about 7 seconds, about 8 seconds, about 9 seconds, about 10 seconds, or more.

In some embodiments, the second fused latent vector 1270 may comprise relevant information from the third latent vector 1268. In some embodiments, the relevant information may comprise relevant information as described with reference to FIG. 2A.

While three latent vectors are illustrated in FIG. 12, it will be appreciated that the processes described herein can be applied to more than three latent vectors.

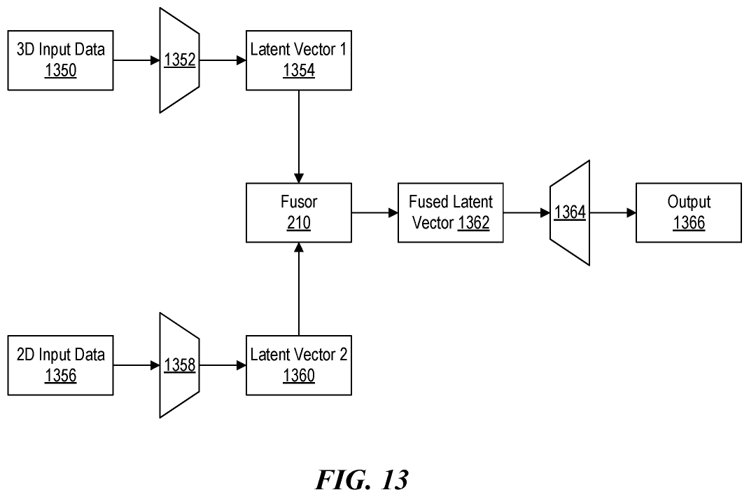

FIG. 13 illustrates a method and associated components of a process for using two-dimensional and three-dimensional data. In some embodiments, the process can be used during a surgical procedure. In some embodiments, two-dimensional can include, for example, data from an SLO imaging device. In some embodiments, three-dimensional data can include, for example, OCT imaging data. Other imaging modalities are possible. For example, the two-dimensional data can include radiography, MRI, CT, ultrasound, elastography, photoacoustic imaging, tomography, x-ray imaging, and/or any other two-dimensional data, for example any other two-dimensional medical imaging data. In some embodiments, three-dimensional data can include x-ray, CT, MRI, tomosynthesis, tomography, and/or any other three dimensional data, for example any other three-dimensional medical imaging data.

In some embodiments, a system can receive 3D input data 1350 at a first time. In some embodiments, the first time may comprise a start of the treatment. In some embodiments, the first time may comprise a time before the start of the treatment, for example, a visit with the clinician before a day of the treatment, the day of the treatment when the patient is being prepped for treatment, when the patient is in the operating room or procedure room before the start of treatment, etc. In some embodiments, the first time may comprise any time throughout the treatment procedure. In some embodiments, the system can generate a first latent vector 1354 from the 3D input data 1350 using an encoder 1352. The encoder 1352 can be trained to process three-dimensional data.

In some embodiments, the system can receive 2D input data 1356 at a second time. In some embodiments, the second time can be after the first time. In some embodiments, the second time can be the same or substantially the same as the first time. In some embodiments, the second time may comprise a time after the first time. In some embodiments, the second time may comprise a time of about 0.1 ms, about 0.5 ms, about 1 ms, about 2 ms, about 3 ms, about 4 ms, about 5 ms, about 6 ms, about 7 ms, about 8 ms, about 9 ms, about 10 ms, about 11 ms, about 12 ms, about 13 ms, about 14 ms, about 15 ms, about 16 ms, about 17 ms, about 18 ms, about 19 ms, about 20 ms, about 21 ms, about 22 ms, about 23 ms, about 24 ms, about 25 ms, about 26 ms, about 27 ms, about 28 ms, about 29 ms, about 30 ms, about 31 ms, about 32 ms, about 33 ms, about 34 ms, about 35 ms, about 36 ms, about 37 ms, about 38 ms, about 39 ms, about 40 ms, about 41 ms, about 42 ms, about 43 ms, about 44 ms, about 45 ms, about 46 ms, about 47 ms, about 48 ms, about 49 ms, about 50 ms, about 60 ms, about 70 ms, about 80 ms, about 90 ms, about 100 ms, about 150 ms, about 200 ms, about 250 ms, about 300 ms, about 350 ms, about 400 ms, about 450 ms, about 500 ms, about 550 ms, about 600 ms, about 650 ms, about 700 ms, about 750 ms, about 800 ms, about 850 ms, about 900 ms, about 950 ms, about 1 second, about 2 seconds, about 3 seconds, about 4 seconds, about 5 seconds, about 6 seconds, about 7 seconds, about 8 seconds, about 9 seconds, about 10 seconds, about 20 seconds, about 30 seconds, about 40 seconds, about 50 seconds, about 1 minute, about 2 minutes, about 3 minutes, about 4 minutes, about 5 minutes, about 6 minutes, about 7 minutes, about 8 minutes, about 9 minutes, about 10 minutes, about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, and/or any other time between the aforementioned times after the first time.

In some embodiments, the system can generate a second latent vector 1360 from the 2D input data 1356 using an encoder 1358. In some embodiments, the encoder 1358 can be configured to process two-dimensional data.

In some embodiments, the output 1366 can comprise a reconstruction of input data captured from a 3D data source at the second time. In some embodiments, the output 1366 may comprise a prediction or representation of what the input data would be if the three-dimensional data source captured input data at the second time.

In some embodiments, a time between the second time and a time when the decoder 1364 generates an output 1366 from the fused latent vector 1362 can be less time than a time the three-dimensional data source would take to capture additional three-dimensional data. For example, the time to reconstruct an OCT image based on an OCT image captured at a first time and an SLO image captured at a second time can be less than the time it would take to capture two OCT images.

### Target-Specific Latent Vectors

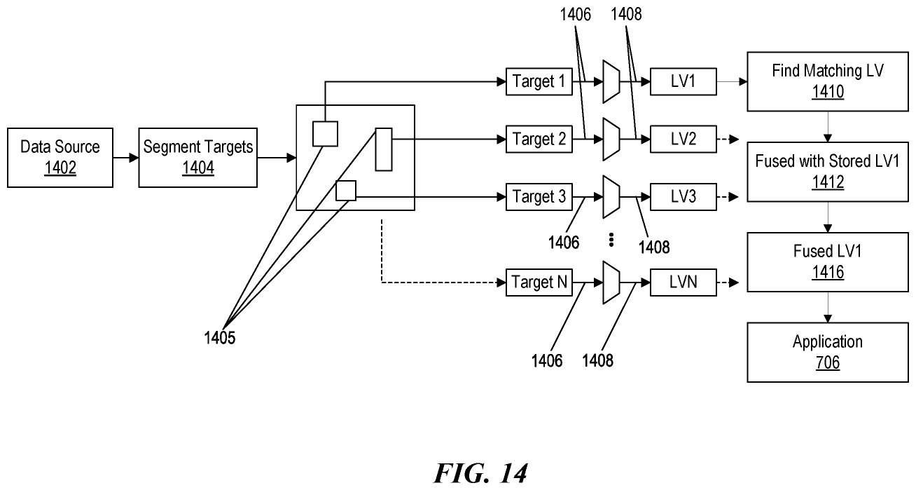

FIG. 14 schematically illustrates a method 1400 for generating target-specific latent vectors according to some embodiments. In some embodiments, the system can receive data from a data source 1402. In some embodiments, the data can comprise, for example, a 2D image or a 3D image. At operation 1404, the system can segment the received data into a plurality of segments 1405. In some embodiments, each segment 1405 can comprise a target. In some embodiments, a target can include a portion of an image including one or more predetermined features or indications of a condition. For example, a target can include a portion of an image including a floater, a floater shadow, one or more cells, a wound, and/or any other feature or indication of a condition.

FIG. 13 shows a schematic of a method for generating target-specific latent vectors 208. In some embodiments, at operation 1302, a data source 104 can capture input data at a first time. In some embodiments, the data source 104 can transmit the input data to the model 200.

In some embodiments, at operation 1304, the model 200 can segment or parse the input data into a plurality of segments 1305. In some embodiments, each of the plurality of segments 1305 may comprise a target. In some embodiments, a target may comprise a portion of an image including one or more predetermined features or indications of a condition. For example, the target may comprise a portion of an image including a floater, one or more cells, a wound, and/or any other feature or indication of a condition. At operation 1406, the system can provide each target to an encoder which, at operation 1408, can output a latent vector for each target. For the sake of readability, FIG. 14 and this description relate to a single latent vector for a single target. However, it will be appreciated that each latent vector can be treated in the same or a similar manner.

In some embodiments, the system can analyze the first latent vector to determine a matching latent vector in a latent vector data store. In some embodiments, the matching latent vector can be, for example, a latent vector generated at a previous time. In some embodiments, the latent vector can correspond to the same target as the first latent vector.

In some embodiments, at operation 1412, a system can use a fusor (for example as described herein) to fuse, merge, unify, join, or otherwise combine the first latent vector and the matching latent vector in the latent vector data store. In some embodiments, the system can, using the fusor, generate a fused first latent vector 1416. In some embodiments, the fused first latent vector 1416 can include or can represent relevant information from the first latent vector and the matching latent vector in the latent vector data store.

In some embodiments, the fused latent vector 1416 can be provided to an application 706. In some embodiments, the application 706 may comprise an application configured to generate a three-dimensional output, generate a treatment plan, annotate the target in the first segment, generate instructions for a surgical device, and/or any other clinical or surgical task or function.

As described herein, operations 1410-1416 can be performed for each latent vector. In some embodiments, processing can occur serially (e.g., latent vectors can be processed one after another). In some embodiments, processing can occur in parallel.

In some embodiments, the same encoder can be used to process each target. In some embodiments, different encoders can be used to process different targets.

In some embodiments, the process described in FIG. 14 can be carried out each time new data is acquired, for example each time a new image frame, portion of an image, etc., is acquired.

### Image Sampling

As described herein, different imaging modalities can have substantially different acquisition times. For example, 2D SLO imaging can be significantly faster than 3D OCT imaging. In some cases, scanning 3D volumes can be too slow for accurate tracking during treatment. For example, an OCT scan can take about one second to capture an entire volume, which can be too slow to track a moving floater during a procedure.

In some cases, scan speeds can be increased. However, there can be physical limitations on scan speed. For example, a minimum integration time may be needed to collect sufficient photons for accurate imaging. While more sensitive photodetectors can reduce scan times, the time required to scan an entire volume can still be too long to enable tracking on short time scales (e.g., real time or nearly real time tracking).

In some embodiments, as described herein, a volume can be sampled rather than scanning the entire volume. In some embodiments, samples can be combined and used to reconstruct the entire volume. In some embodiments, multiple imaging modes can be used. For example, a first imaging modality (e.g., SLO) can be used to capture an image within a field of view. In some embodiments, there can be a region of interest in the field of view, for example a shadow that may indicate the presence of a floater. In some embodiments, an imaging processing algorithm can be configured to identify one or more regions of interest in the first image. In some embodiments, a second imaging modality (e.g., OCT) can be used to collect additional information. In some embodiments, the second imaging modality can use sampling, for example as opposed to scanning an entire field of view to capture a frame.

In some embodiments, a computer system can be configured to generate a sampling plan. For example, in some embodiments, a sampling plan can include grid sampling or random sampling. In some embodiments, sampling can be based on, for example, a feature or region of interest. For example, if a first imaging modality shows a region of interest (e.g., a floater shadow), the system can be configured to generate a sampling plan that samples within the region of interest.

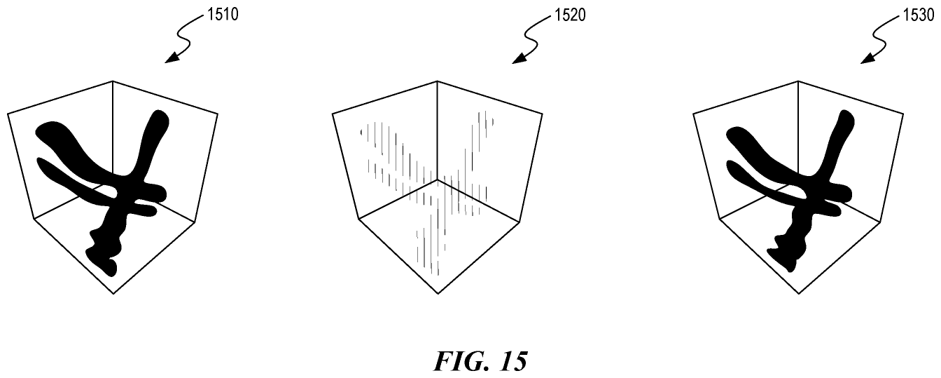

FIG. 15 schematically illustrates image reconstruction according to some embodiments. In FIG. 15, the scan 1510 can indicate a scan of an entire volume. In some embodiments, instead of scanning the entire volume, the volume can be sampled. The scan 1520 shows a sparse sampling scan according to some embodiments. The scan 1520 samples only a fraction of the full volume. In some embodiments, an AI/ML model can be configured to reconstruct a full scan based on a sparse scan, such as the scan 1520. The reconstructed scan 1530 illustrates an example of a scan reconstruction based on the sampled scan 1520. As shown in FIG. 15, the reconstructed scan 1530 can generally capture the features of the full scan 1510, although there can be some differences. How closely the reconstructed scan mirrors the full scan 1510 can depend on, for example, a completeness of the sparse scan, a scanning pattern of the sparse scan, etc. For example, the reconstructed scan 1530 can improve if a denser sampling is obtained. However, denser sampling can be associated with increased imaging times. In some embodiments, a sparse scanning pattern can be designed to sample more heavily in a region of interest, which can improve reconstructed image quality.

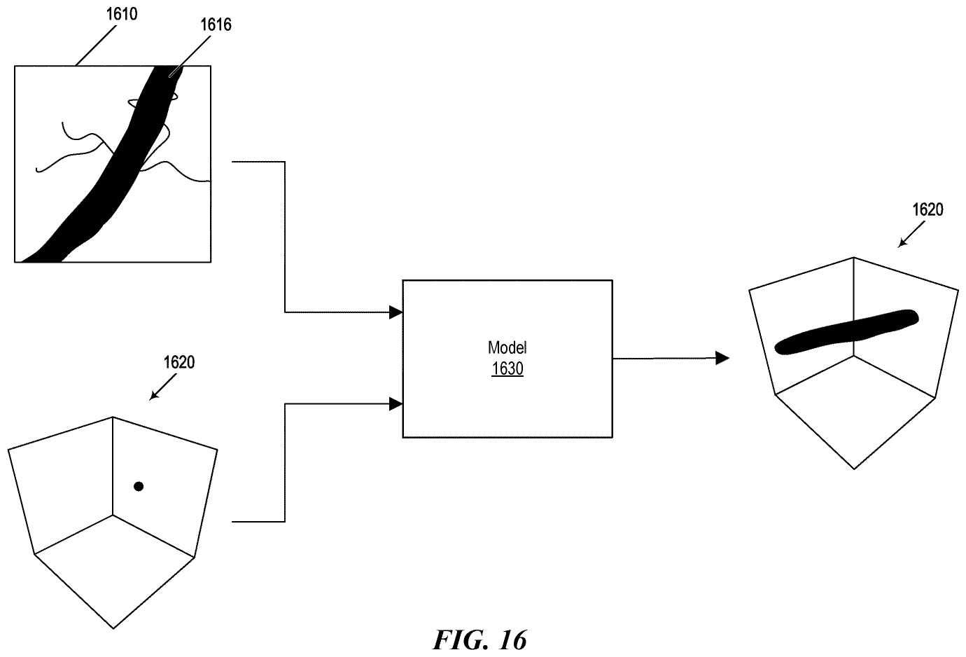

FIG. 16 is a drawing that schematically illustrates combining two imaging modalities according to some embodiments. In FIG. 16, a first image 1610 (e.g., an SLO image) can include a region of interest 1616. The region of interest 1616 can be, for example, a floater shadow. A system can be configured to identify the region of interest 1616. In some embodiments, a user can select a region of interest using a graphical user interface. In some embodiments, the system can perform a sparse scan 1620. The sparse scan 1620 can sample a small portion of a volume, for example about 1% of a volume. The sparse scan 1620 can include scanning at least one point or collection of points within the region of interest 1616. The first image 1610 and the sparse scan 1620 can be provided to a machine learning model 1630. The machine learning model 1630 can be configured to generate a first latent vector for the first image 1610 and a second latent vector for the sparse scan 1620. The system can fuse the first latent vector and the second latent vector. The system can use the fused latent vector to generate a reconstructed scan 1640. It will be appreciated that the sparse scan 1620 can include one sample or a plurality of samples. In some embodiments, each sample of the plurality of samples can be encoded to create a plurality of latent vectors, which can be fused to generate a fused latent vector as described herein. This fused latent vector can be fused first latent vector for the first image 1610 by the machine learning model 1630.

The reconstructed scan 1640 can, in some embodiments, indicate what a full scan would look like based on the sparse scan and the first image 1610. For example, if the first image is an SLO image and the sparse scan is an OCT scan, the reconstructed scan 1640 can indicate what an OCT image would look like if the entire volume had been scanned, rather than only a portion. In some embodiments, the sparse scan can scan, for example, about 1% of the volume. In some embodiments, the sparse scan can scan less than 1% of the volume. In some embodiments, the sparse scan can scan more than 1% of the volume. In some embodiments, the amount of the volume that is scanned can vary based on, for example, an intended use of the reconstructed scan 1640 (e.g., for screening, diagnostics, surgical planning, surgical treatment, etc.). In some embodiments, a user can customize a threshold or otherwise configure how much of the volume should be scanned to generate the sparse scan 1620.

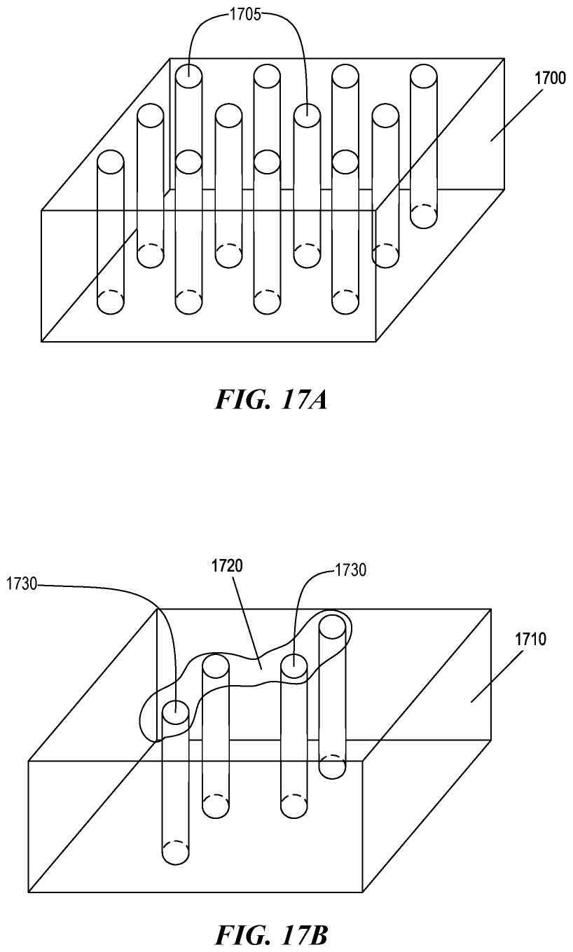

FIG. 17A shows an example of sparse scanning according to some embodiments. In FIG. 17A, a volume 1700 is scanned at sampling locations 1705. In FIG. 17A, only two sampling locations 1705 are labeled for the sake of clarity and ease of understanding, but there can be more sampling locations, as shown in FIG. 17A. In FIG. 17A, the sampling locations 1705 are distributed in an organized pattern through the volume 1700. However, other implementations are possible. For example, in some embodiments, the sampling locations 1705 can be randomly distributed.

In some embodiments, sampling locations can be adjusted in real time or substantially real time. For example, if a previous scan location (e.g., a previous random scan location) identifies a feature of interest, subsequent scans can be made to be non-random and instead scan a pattern of locations based on the feature of interest.

As described herein, in some embodiments, sampling locations can be selected based on the presence of a region of interest. FIG. 17B schematically illustrates sparse scanning according to some embodiments, in which the sampling locations are selected based on a region of interest. In some embodiments, a region of interest can be identified using a different imaging modality, an earlier scan with a same imaging modality, etc. In some embodiments, the ROI can be identified in, for example, an SLO image. In FIG. 17B, a volume 1710 is sparsely scanned at sampling locations 1730. The sampling locations fall within a region of interest 1720. The region of interest 1720 can be, for example, based on a region of interest identified in another image.

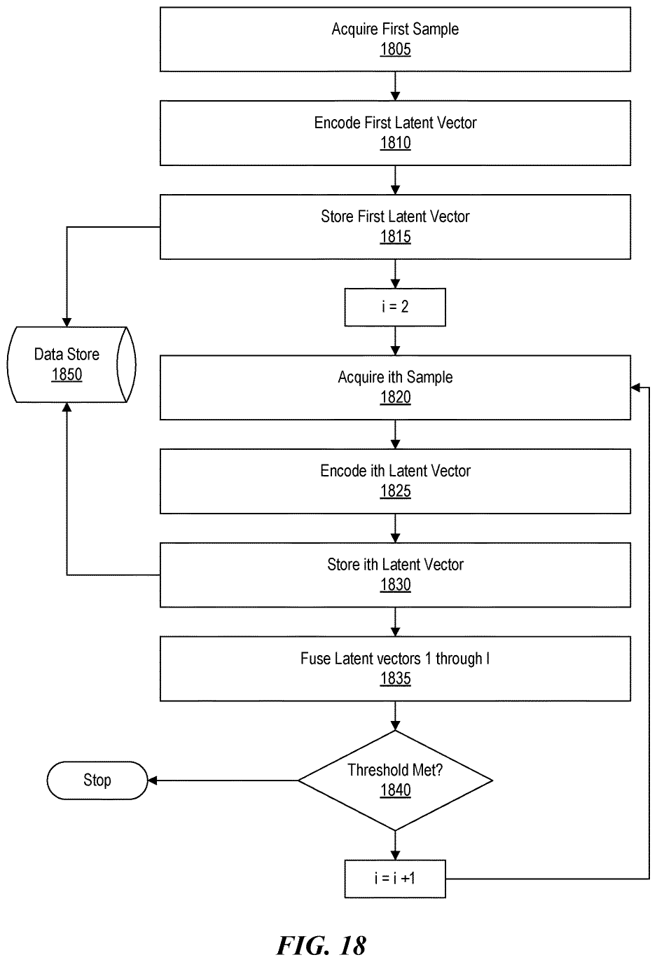

FIG. 18 is a flowchart that illustrates an example sparse scanning process according to some embodiments. The process 1800 can be implemented as a computer-implemented method that can be carried out on one or more computer systems.

At operation 1805, a system can acquire a first sample. The first sample can be an image of a portion of an area or volume. At operation 1810, the system can encode a first latent vector based on the first sample. At operation 1815, the system can store the first latent vector in a data store 1850. The system can collect additional samples. At operation 1820, the system can acquire a next sample. At operation 1825, the system can encode the next sample to generate a next latent vector. At operation 1830, the system can store the next latent vector in the data store 1850. At operation 1835, the system can fuse the latent vector and the next latent vector to generate a fused latent vector. At operation 1840, the system can determine if one or more thresholds (e.g., a completion threshold) are met. If so, the process can stop. If not, the process can continue at operation 1820. The number of samples that can be collected is not necessarily limited. In some embodiments, the number of samples can be set to a maximum number of samples. In some embodiments, sampling can stop once a threshold is met. For example, once a change in the fused latent vector from one iteration to the next changes by less than a threshold amount, the system can stop collecting additional samples. In some embodiments, the threshold can be fixed. In some embodiments, a user can adjust a threshold, which can cause the system to collect more samples (if the threshold for minimum change in the fused latent vector is set lower) or fewer samples (if the threshold for minimum change in the fused latent vector is set higher). In some embodiments, the threshold can vary depending upon the application or use for the fused latent vector. For example, when used for screening or diagnosis, a threshold may be lower than if the fused latent vector is to be used during surgery to make treatment decisions.

It will be appreciated that fusing latent vectors can occur in a variety of ways. For example, in some embodiments, at operation 1835, the system can fuse each acquired latent vector. In some embodiments, at operation 1835, the system can fuse a newly acquired latent vector with a previously generated fused latent vector.

### Imaging and Surgical Hardware Control

Image capture typically uses well-known raster scanning techniques, in which an image frame is captured by scanning from a starting point to an ending point in a regular pattern, such as by tracing out a path from left to right and top to bottom. While this can work well for certain applications, difficulties can be encountered when there is fast movement. For example, when a scan begins at the top of a frame and proceeds towards the bottom, a top portion of the captured image may not accurately show the current position of a feature (e.g., a floater), as the object may have moved.

In some cases, capture of a full frame may not be necessary. For example, if an image is being captured for purposes of object pose detection, there may be no need for a full frame and it may be more important that information throughout a field of view be captured quickly. For example, if imaging is used to determine the path, treatment locations, or both of a treatment laser, it can be important to know where a target object (e.g., a floater) is located in real time or nearly real time, but there may be little or even no need for a full image capture.

In some embodiments, rather than performing a traditional raster scan, a system can be configured to scan in another pattern, such as a spiral or any other arbitrary pattern. Typically, scanning lasers and treatment lasers are controlled via galvanometers, which can allow scanning along arbitrary paths. By scanning along a path such as a spiral, image data can be captured throughout a field of view quickly, allowing faster determination of the location, orientation, etc., of objects within the field of view, which can improve treatment outcomes as, for example, the path of a treatment laser, locations for treatment, or both can be adjusted more quickly based on information that more accurately represents a current position of the target object. In some embodiments, scanning can be continuous, for example sweeping out a first path following generally from an edge of the field of view to the center of the field of view, followed by scanning another path from the center of the field of view toward the outer edges of the field of view.

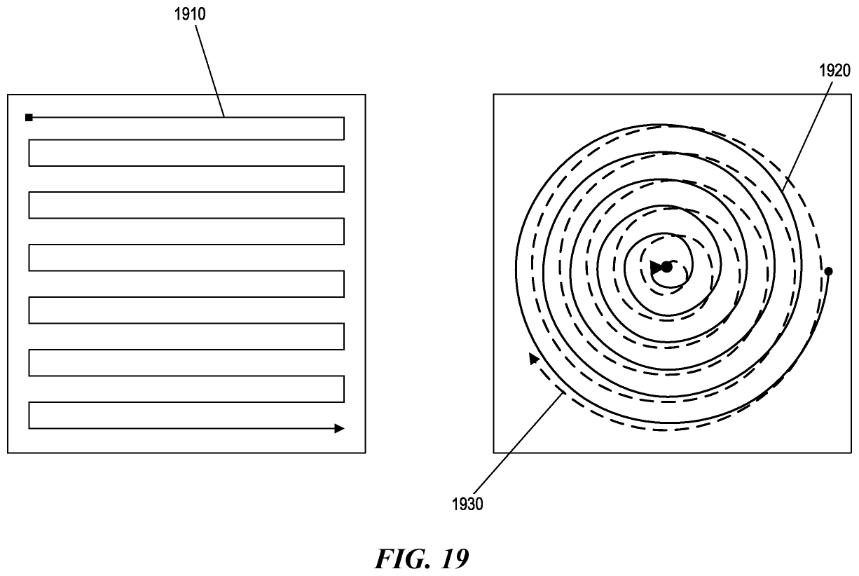

FIG. 19 shows examples of scanning paths according to some embodiments. The path 1910 shows a typical raster scanning path, tracing out horizontal and vertical lines that go from a top left corner of the field of view to the bottom right corner of the field of view. As described herein, this can have disadvantages as, for example, an object in the field of view may have moved between the time a top left sample was captured and the time a bottom right scan was captured, which can lead to errors, uncertainty, or both in the position, size, etc., of an object in the field of view. In some embodiments, different paths can be selected. For example, the path 1920 follows a spiral shape from near an outer edge of the field of view toward the center of the field of view. Thus, image information can be captured across distant regions of the field of view more quickly than if raster scanning were performed. The path 1930 can follow generally from the center to the edge in a spiral pattern. Thus, for example, scanning patterns can change from frame to frame. However, it will be appreciated that, as described herein, full frame capture may not be necessary in some applications.

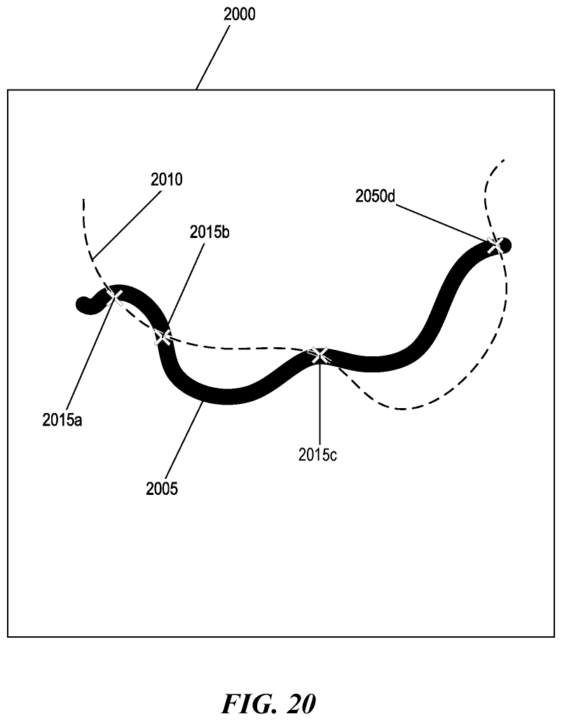

FIG. 20 is a drawing that schematically illustrates an example laser path and treatment points according to some embodiments. In FIG. 20, a floater shadow 2005 is present in a field 2000. A treatment laser can trace out a path 2010 which can cross the shadow 2005. The treatment laser can fire at treatment points 2005a, 2005b, 2005c, 2005d, which can break up a floater associated with the floater shadow 2005. The treatment path 2010 can be determined based on, for example, imaging data (e.g., sparse OCT imaging data, SLO data, and/or the like). In some embodiments, a scanning laser and a treatment laser can be controlled by the same galvanometers. In some embodiments, the treatment laser can follow the scanning laser. In some embodiments, a system can be configured to use the fused latent vector techniques described herein to rapidly determine a position of a floater, such that a following treatment laser can apply pulses in targeted areas before the floater moves significantly.

As shown in FIG. 20, a treatment laser may not apply treatment continuously. For example, a YAG laser can be a pulsed laser that can be triggered whenever the laser crosses a treatment point.

### Morphology and Pose Separation

Floaters can have relatively static morphology, at least over relevant time scales. However, floaters are typically in constant or nearly constant motion. When carrying out a treatment procedure, it can be important to know both the pose (e.g., position in x, y, and z dimensions, as well as pitch, yaw, and roll) and the morphology. For example, knowing the current pose of a floater can be important for determining a treatment plan, directing a laser to provide treatment, and so forth. In some embodiments, there can be an N: 1 ration of pose to morphology. That is, for example, for a given morphology, there can be N poses, where N can be any positive integer.

In some embodiments, when an image of a floater is captured, the imaging data can be encoded into separate morphology information and pose information. In some embodiments, morphology can be determined by accumulating morphology data until an accuracy threshold has been reached.

In some embodiments, a morphology latent vector can be associated with a particular floater. In some embodiments, the morphology latent vector can include a label or can have a label associated therewith, where the label can be used to identify the floater. By associating each floater with a label, screening and/or diagnosis can be carried out even when data acquisition speed is not an issue.

In some embodiments, pose can be updated at regular intervals. However, less information may be needed to resolve pose. Thus, in some embodiments, pose information can be updated based on sparse scanning, which can enable faster acquisition and more frequent pose updates. In some embodiments, morphology can be tied to treatment patterns.

In some embodiments, a system can be configured to acquire a data sample. In some embodiments, the data sample can be an OCT image. In some embodiments, the sample can be a sparse sample, for example a sparse OCT image. In some embodiments, the data sample can be used to generate a pose latent vector and a morphology latent vector. In some embodiments, during earlier captures, pose can be ignored because morphology is not yet well-defined. In some embodiments, for a number of timesteps T, the system can be configured to capture additional samples. The additional samples can be used to encode additional morphology latent vectors, which can be fused with previous morphology latent vectors or previous morphology fused latent vectors. In some embodiments, the collection and fusing process can continue until the morphology is determined within a threshold, for example when the fused morphology latent vector does not change by more than a predetermined amount when new samples are added.

In some embodiments, after the threshold is reached, at each new timestep, the pose latent vector can be used to predict the orientation of the floater based on a small number of samples and the previously determined morphology.

Such an approach can enable accurate 3D tracking of an object, such as a floater, based on sparse data. In some embodiments, a first set of samples can be capture at a first density (e.g., full scans) and, once the morphology of the object has been determined, sparse sampling can be used for tracking the object.

During treatment, morphology and pose may have already been defined or can be measured prior to treatment. In some embodiments, morphology may have been determined at an earlier point in time, for example when a patient was screened or diagnosed. In some embodiments, during a treatment procedure, a system can be configured to determine if the pose of a floater is not changing or if the rate of change is below a threshold amount. If the floater is stationary and sufficiently stable, the system can cause treatment to be performed, such as directing a treatment laser at the floater. In some embodiments, after the treatment is applied (e.g., after the treatment laser is used), the system can acquire an updated morphology of the floater. In some embodiments, the original morphology and the new morphology can be used to detect a change in morphology following treatment. The change can help to inform, for example, whether additional treatment is likely to be beneficial to the patient.

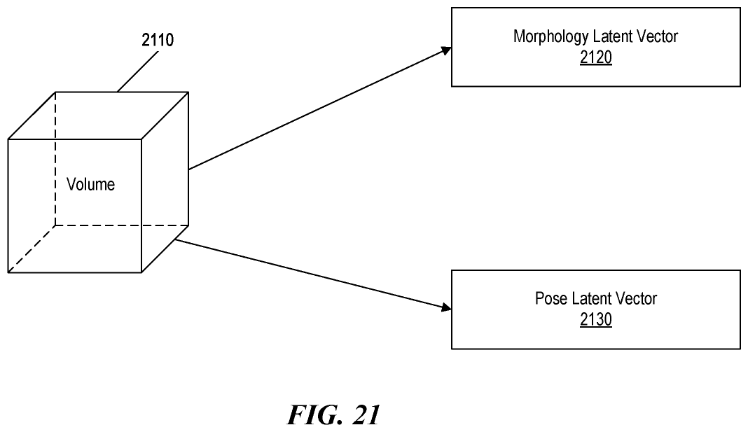

FIG. 21 is a drawing that illustrates morphology and pose separation according to some embodiments. A volume 2110 can be scanned (e.g., fully scanned or sparsely scanned), and the resulting data can be used to generate a morphology latent vector 2121 and a pose latent vector 2130.

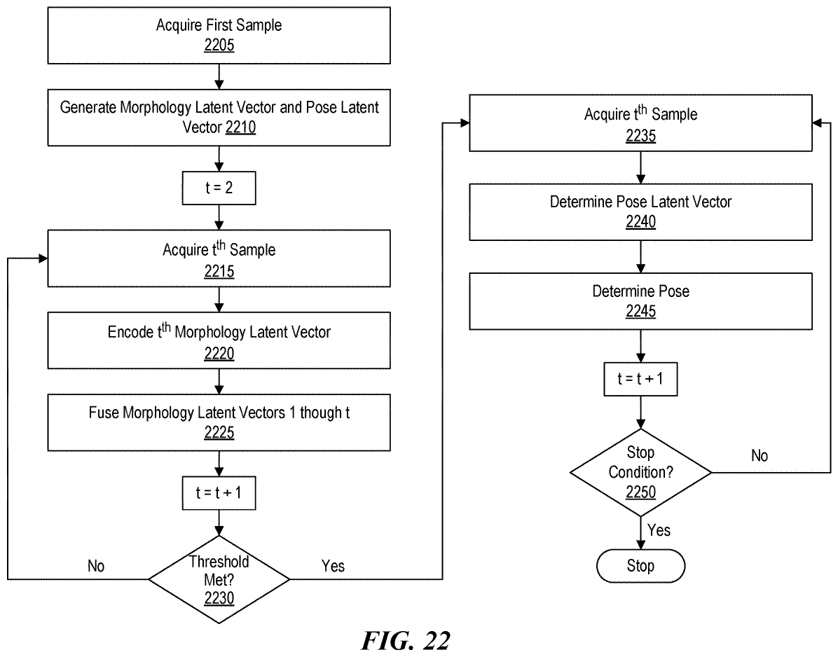

FIG. 22 is a flowchart that illustrates an example process for generating pose and morphology latent vectors according to some embodiments. At operation 2205, a system can acquire a first sample. At operation 2210, the system can generate a morphology latent vector and a pose latent vector. In some embodiments, the system can instead load an initial morphology latent vector, an initial pose latent vector, or both. During initial iterations, the pose vector may not be updated. For example, it can be important to have a well-defined morphology before determining pose. At a t^{th} time step (for t greater than or equal to 2), the system can, at operation 2215, acquire a t^{th} sample. At operation 2220, the system can encode a t^{th} morphology latent vector. At operation 2225, the system can fuse morphology latent vectors 1 through t. In some embodiments, a t^{th} latent vector can be fused with a fused latent vector generated from fusing latent vectors from previous samples (e.g., 1 through t−1). For example, for t=3, the third latent vector can be fused with a fused latent vector that was generated by fusing the first and second latent vectors. At operation 2230, the system can determine if a threshold criterion has been satisfied. For example, if the morphology latent vector changes by less than a threshold amount between one iteration and the next, the threshold can be met and the process can continue. It will be appreciated that the threshold can vary depending upon the application. Lower thresholds may be used when more precise morphology information is desirable, for example when performing a treatment procedure.

As depicted in FIG. 22, determining if a threshold is met at operation 2230 occurs after at least two samples are acquired. However, in some embodiments, checking if the threshold is satisfied can be done based on a single sample. For example, if there is only one sample acquired, or information is loaded from storage, in some embodiments, checking for passing the threshold can be performed at t=1.

The relationship between morphology and pose can have significant implications. A single morphology can have many different possible poses. This can have implications for storage, retrieval, updates, etc. For example, less storage space may be needed if pose and morphology are separated, as there may not be a need to store morphology data alongside each representation of pose. In some embodiments, there can be different thresholds for morphology and pose.

At operation 2235, the system can acquire a t^{th} sample. The system can, at operation 2240, determine a pose latent vector. At operation 2245, the system can determine a pose based on the pose latent vector. Operations 2235 through 2245 can be repeated until a stop condition is met at operation 2250. A stop condition can be, for example, a maximum acquisition time being reached, a procedure being finished, or the like. It will be appreciated that, generally, less information may be needed to determine pose than to determine morphology. Thus, in some embodiments, scanning during the pose determination phase may be sparser than scanning performed during the morphology determination phase, although this is not necessarily the case.

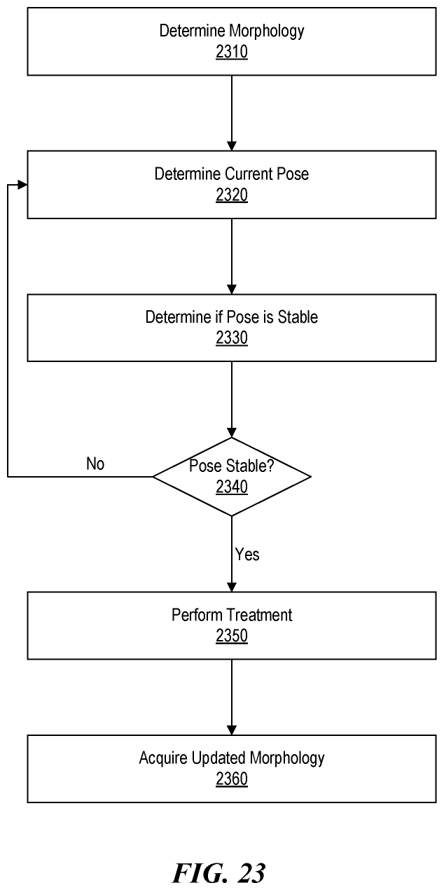

FIG. 23 is a flowchart that illustrates an example process for treating a patient according to some embodiments. The operation can be carried out on a computer system or multiple computer systems (generally “system”). At operation 2310, the system can determine morphology of a target object (e.g., a floater), for example as described herein. At operation 2320, the system can determine a current pose of the target object, for example as described herein. At operation 2330, the system can determine if the pose is stable, for example by comparing the current pose to a previously-determined pose at an earlier time. At operation 2340, if the pose is not stable, the system can continue determining the pose of the target object. If the pose of the target object is stable, the process can continue to operation 2350 and perform a treatment, for example by directing a pulsed laser along a path and directed the laser to trigger such as energy is directed into the target object. After treatment, the system can, at operation 2360, acquire an updated morphology of the target object.

In some embodiments, the updated morphology can be compared to the morphology of the target object prior to treatment, which can be used to gauge the effectiveness of the treatment and/or to determine if additional treatment should be performed, either during a current procedure or at a second procedure to be carried out at a future date.

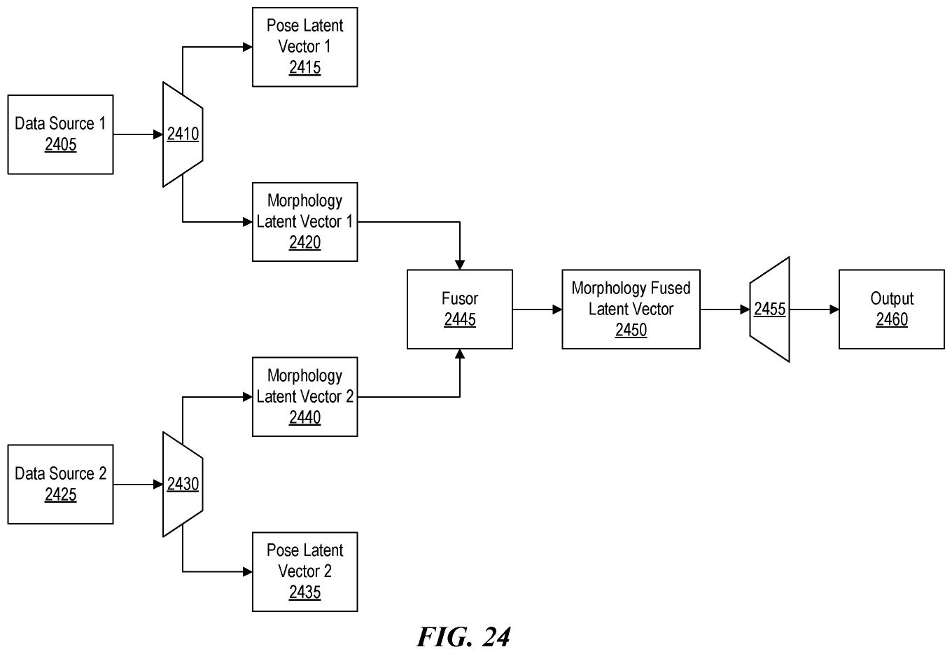

FIG. 24 is a diagram that illustrates an example of generating and processing pose and morphology latent vectors according to some embodiments. The process shown in FIG. 24 can be performed by a computer system or multiple computer systems (generally “system”). A first encoder 2410 can receive information from a first data source 2405. The first encoder 2410 can generate a first pose latent vector 2415 and a first morphology latent vector 2420. A second encoder 2430 can receive information from a second data source 2425. In some embodiments, the second data source 2425 can be the same data source as the first data source 2405. The second encoder 2430 can generate a second pose latent vector 2435 and a second morphology latent vector 2440. A fusor 2445 can receive the first morphology latent vector 2420 and the second morphology latent vector 2440 and can generate a fused morphology latent vector 2450. A decoder 2455 can receive the fused morphology latent vector 2450 and generate an output 2460.

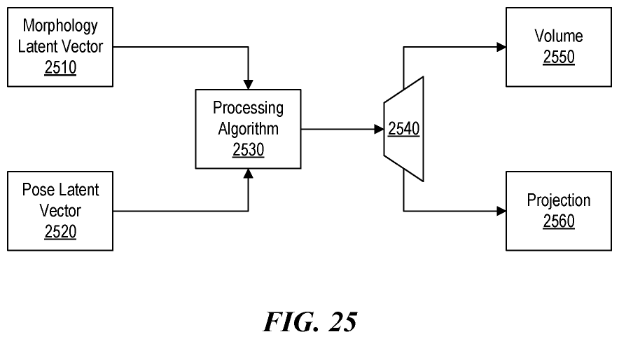

In some embodiments, morphology latent vectors and pose latent vectors can be provided to a processing algorithm for further application. FIG. 25 illustrates an example of using morphology latent vectors and pose latent vectors according to some embodiments. A processing algorithm 2530 can receive a morphology latent vector 2510 and a pose latent vector 2520. The processing algorithm 2530 can provide an output to a decoder 2540. The decoder 2540 can generate one or more outputs. For example, in FIG. 25, the decoder 2540 generates a volume output 2550 and a projection output 2560. The volume output 2550 can be, for example, a volumetric image. The projection output 2560 can be, for example, an SLO image (e.g., a generated SLO image). While one decoder is depicted in FIG. 25, in some embodiments, there can be multiple decoders. For example, a first decoder can produce the volume output 2550 and a second encoder can produce the projection output 2560.

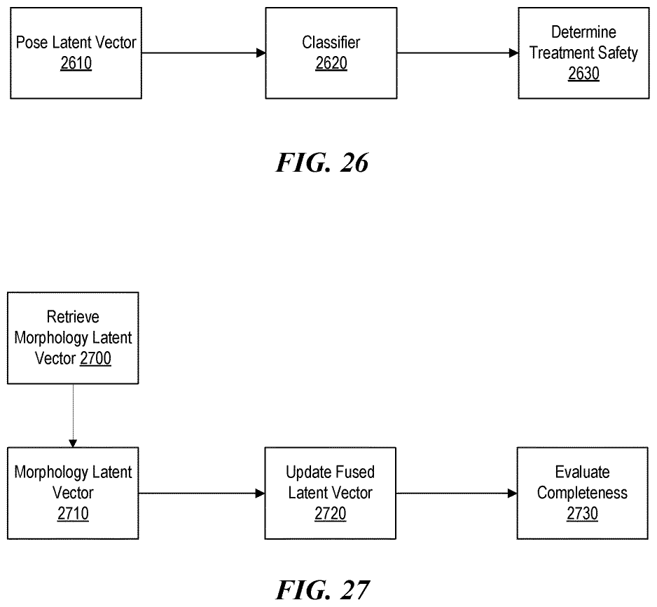

FIG. 26 shows an application for a pose latent vector according to some embodiments. A pose latent vector 2610 can be provided to a classifier model 2620. Based on the output of the classifier model 2620, a system can determine treatment safety 2630. For example, treatment safety for a floater can depend on the location of the floater, orientation of the floater, etc.

FIG. 27 illustrates a process for handling morphology latent vectors according to some embodiments. As described herein, a significant amount of data can be needed to determine morphology of a target object such as a floater. This can be especially true for target objects that tend to move, that have irregular shapes, etc. At operation 2700, a system can receive a morphology latent vector 2710. In some embodiments, the system can download and store the morphology latent vector 2710. In some embodiments, the system can retrieve the morphology latent vector 2710 from cloud storage. In some embodiments, the system can retrieve the morphology latent vector 2710 using an API. The system can, at operation 2720, updated a fused latent vector using the morphology latent vector 2710. The fused latent vector can be, for example, a morphology latent vector generated from previously-generated morphology latent vectors. At operation 2730, the system can evaluate completeness of the fused latent vector, for example by comparing the updated fused latent vector to a previous fused latent vector to determine if a change in the fused latent vector is greater than or less than a threshold amount.

### Adaptive Floater Screening

In current approaches, OCT imaging and SLO imaging are decoupled. That is, scans for one imaging modality (e.g., OCT) are not informed by information from the other imaging modality (e.g., SLO).

In some embodiments, results from a first imaging modality may not be above a confidence threshold. In some embodiments, a system can be configured to automatically or dynamically use a second imaging modality to improve confidence. In some embodiments, the second imaging modality can be a modality that collects more information but does so in a more limited field of view. Such an approach can improve confidence.

As an example, an SLO scan can detect multiple floater shadows. Some shadows can be above a confidence threshold for being confirmed as floaters, while other shadows can be below the confidence threshold. In some embodiments, a system can be configured to use OCT to scan areas where shadows are present that are below the confidence threshold. In some embodiments, sparse OCT scanning, for example as described herein, can be used. The OCT scanning can be used to increase confidence in determining whether or not a shadow corresponds to a floater. For example, the OCT scan can cause a shadow below the confidence threshold to be confirmed as corresponding to a floater or can cause a shadow below the confidence threshold to be confirmed as not corresponding to a floater.

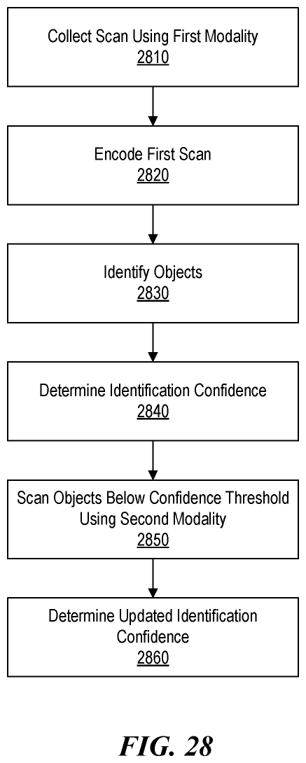

FIG. 28 is a flowchart that illustrates an example process for updating identification confidence according to some embodiments. The process illustrated in FIG. 28 can be carried out on a computer system or multiple computer systems (generally “system”).

At operation 2810, the system can collect a scan using a first modality (e.g., SLO). At operation 2820, the system can encode the first scan to generate a latent vector. At operation 2830, the system can identify one or more objects in the first scan, for example using the latent vector. At operation 2840, the system can determine an identification confidence for each identified object in the first scan. For example, it may not be clear if an identified object is a floater, imaging artifact, feature of the eye, etc. At operation 2850, the system can scan one or more objects below a confidence threshold using a second modality (e.g., OCT). At operation 2860, the system can, based at least in part on the second modality scan (e.g., using a latent vector of the second modality scan), determine an updated identification confidence for each of the one or more objects below the confidence threshold.

### Computer Systems

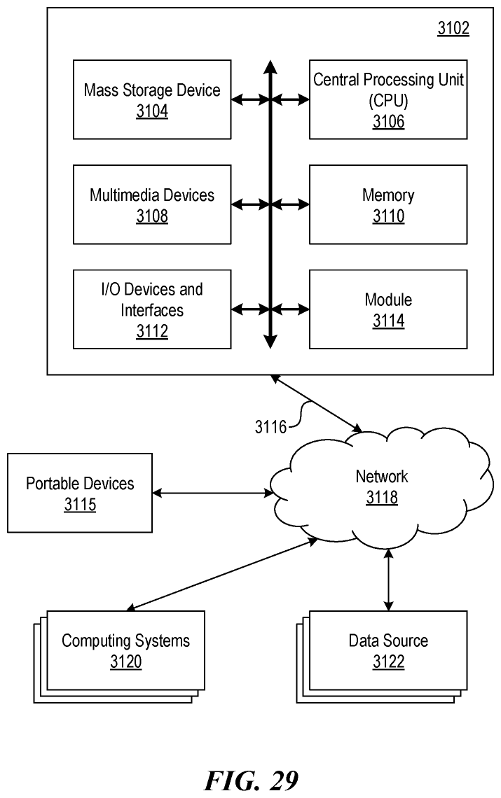

FIG. 29 is a block diagram depicting an embodiment of a computer hardware system configured to run software for implementing one or more embodiments disclosed herein.

In some embodiments, the systems, processes, and methods described herein are implemented using a computing system, such as the one illustrated in FIG. 29. The example computer system 3102 is in communication with one or more computing systems 3120 and/or one or more data sources 3122 via one or more networks 3118. While FIG. 13 illustrates an embodiment of a computing system 3102, it is recognized that the functionality provided for in the components and modules of computer system 3102 may be combined into fewer components and modules, or further separated into additional components and modules.

The computer system 3102 can comprise a module 3114 that carries out the functions, methods, acts, and/or processes described herein. The module 3114 is executed on the computer system 3102 by a central processing unit 3106 discussed further below.

In general, the word “module,” as used herein, refers to logic embodied in hardware or firmware or to a collection of software instructions, having entry and exit points. Modules are written in a program language, such as JAVA, C or C++, Python, or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 3102 includes one or more processing units (CPU) 3106, which may comprise a microprocessor. The computer system 3102 further includes a physical memory 3110, such as random-access memory (RAM) for temporary storage of information, a read only memory (ROM) for permanent storage of information, and a mass storage device 3104, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 3102 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 3102 includes one or more input/output (I/O) devices and interfaces 3112, such as a keyboard, mouse, touch pad, and printer. The I/O devices and interfaces 3112 may comprise one or more display devices, such as a monitor, which allows the visual presentation of data to a user. More particularly, a display device provides for the presentation of GUIs as application software data, and multi-media presentations, for example. The I/O devices and interfaces 3112 can also provide a communications interface to various external devices. The computer system 3102 may comprise one or more multi-media devices 3108, such as speakers, video cards, graphics accelerators, and microphones, for example.

The computer system 3102 may run on a variety of computing devices, such as a server, a Windows server, a Structure Query Language server, a Unix Server, a personal computer, a laptop computer, and so forth. In other embodiments, the computer system 3102 may run on a cluster computer system, a mainframe computer system and/or other computing system suitable for controlling and/or communicating with large databases, performing high volume transaction processing, and generating reports from large databases. The computing system 3102 is generally controlled and coordinated by an operating system software, such as Windows XP, Windows Vista, Windows 7, Windows 8, Windows 10, Windows 11, Windows Server, Unix, Linux (and its variants such as Debian, Linux Mint, Fedora, and Red Hat), SunOS, Solaris, Blackberry OS, z/OS, iOS, macOS, or other operating systems, including proprietary operating systems. Operating systems control and schedule computer processes for execution, perform memory management, provide file system, networking, and I/O services, and provide a user interface, such as a graphical user interface (GUI), among other things.

The computer system 3102 illustrated in FIG. 29 is coupled to a network 3118, such as a LAN, WAN, or the Internet via a communication link 3116 (wired, wireless, or a combination thereof). Network 3118 communicates with various computing devices and/or other electronic devices. Network 3118 is communicating with one or more computing systems 3120 and one or more data sources 3122. The module 3114 may access or may be accessed by computing systems 3120 and/or data sources 3122 through a web-enabled user access point. Connections may be a direct physical connection, a virtual connection, and other connection type. The web-enabled user access point may comprise a browser module that uses text, graphics, audio, video, and other media to present data and to allow interaction with data via the network 3118.

Access to the module 3114 of the computer system 3102 by computing systems 3120 and/or by data sources 3122 may be through a web-enabled user access point such as the computing systems' 3120 or data source's 3122 personal computer, cellular phone, smartphone, laptop, tablet computer, e-reader device, audio player, or another device capable of connecting to the network 3118. Such a device may have a browser module that is implemented as a module that uses text, graphics, audio, video, and other media to present data and to allow interaction with data via the network 3118.

The output module may be implemented as a combination of an all-points addressable display such as a cathode ray tube (CRT), a liquid crystal display (LCD), a plasma display, or other types and/or combinations of displays. The output module may be implemented to communicate with input devices 3112 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 3102 may comprise a physical or logical connection established between a remote microprocessor and a mainframe host computer for the express purpose of uploading, downloading, or viewing interactive data and databases on-line in real time. The remote microprocessor may be operated by an entity operating the computer system 3102, including the client server systems or the main server system, an/or may be operated by one or more of the data sources 3122 and/or one or more of the computing systems 3120. In some embodiments, terminal emulation software may be used on the microprocessor for participating in the micro-mainframe link.

In some embodiments, computing systems 3120 who are internal to an entity operating the computer system 3102 may access the module 3114 internally as an application or process run by the CPU 3106.

In some embodiments, one or more features of the systems, methods, and devices described herein can utilize a URL and/or cookies, for example for storing and/or transmitting data or user information. A Uniform Resource Locator (URL) may comprise a web address and/or a reference to a web resource that is stored on a database and/or a server. The URL can specify the location of the resource on a computer and/or a computer network. The URL may comprise a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs may comprise a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, may comprise data sent from a website and/or stored on a user's computer. This data can be stored by a user's web browser while the user is browsing. The cookies may comprise useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 3102 may include one or more internal and/or external data sources (for example, data sources 3122). In some embodiments, one or more of the data repositories and the data sources described above may be implemented using a relational database, such as Sybase, Oracle, CodeBase, DB2, PostgreSQL, and Microsoft® SQL Server as well as other types of databases such as, for example, a NoSQL database (for example, Couchbase, Cassandra, or MongoDB), a flat file database, an entity-relationship database, an object-oriented database (for example, InterSystems Caché), a cloud-based database (for example, Amazon RDS, Azure SQL, Microsoft Cosmos DB, Azure Database for MySQL, Azure Database for MariaDB, Azure Cache for Redis, Azure Managed Instance for Apache Cassandra, Google Bare Metal Solution for Oracle on Google Cloud, Google Cloud SQL, Google Cloud Spanner, Google Cloud Big Table, Google Firestore, Google Firebase Realtime Database, Google Memorystore, Google MongoDB Atlas, Amazon Aurora, Amazon DynamoDB, Amazon Redshift, Amazon ElastiCache, Amazon MemoryDB for Redis, Amazon DocumentDB, Amazon Keyspaces, Amazon Neptune, Amazon Timestream, or Amazon QLDB), a non-relational database, or a record-based database.

The computer system 3102 may also access one or more databases 3122. The databases 3122 may be stored in a database or data repository. The computer system 3102 may access the one or more databases 3122 through a network 3118 or may directly access the database or data repository through I/O devices and interfaces 3112. The data repository storing the one or more databases 3122 may reside within the computer system 3102.

### Example Clauses

Clause 1. A computer-implemented method for processing image data during surgery, the method comprising: receiving first image data from a first imaging device, wherein the first image data is captured by the first imaging device at a first time; generating, via a pretrained encoder, a first latent vector from the first image data, wherein the first latent vector comprises first information representing the first image data; receiving second image data from a second imaging device, wherein the second image data is captured by the second imaging device at a second time, and wherein the second time is after the first time; generating, via the pretrained encoder, a second latent vector from the second image data, wherein the second latent vector comprises second information representing the second image data; transmitting the first latent vector and the second latent vector to a pretrained fusor; generating, via the pretrained fusor, a fused latent vector comprising fused information, wherein the fused information comprises the first information of the first latent vector, and new information of the second information of the second latent vector, wherein the new information comprises information of the second information that is not in the first information; transmitting the fused latent vector to a pretrained decoder, wherein the decoder is configured to perform a clinical function from the fused information.
Clause 2. The computer-implemented method of Clause 1, wherein the first imaging device and the second imaging device are a same imaging device.
Clause 3. The computer-implemented method of Clause 1, wherein the first imaging device comprises scanning laser ophthalmoscopy (SLO), optical coherence tomography (OCT), x-ray, magnetic resonance imaging (MRI), computed tomography scanning (CT), ultrasound, and/or fluorescein angiography (FA).
Clause 4. The computer-implemented method of Clause 1, wherein the first image data and/or the second image data comprise one or more targets and one or more structures of a patient's body.
Clause 5. The computer-implemented method of Clause 4, wherein the pretrained fusor in configured to incorporate information representing the one or more targets in the fused information.
Clause 6. The computer-implemented method of Clause 4, wherein the pretrained fusor is configured to ignore information representing the one or more structures of the patient's body.
Clause 7. The computer-implemented method of Clause 1, wherein the pretrained fusor is configured to not incorporate redundant information in the fused information, wherein redundant information comprises same information of the first information and the second information.
Clause 8. The computer-implemented method of Clause 1, further comprising: receiving third image data from a third imaging device, wherein the third image data is captured by the third imaging device at a third time, and wherein the third time is after the second time; generating, via the pretrained encoder, a third latent vector from the third image data, wherein the third latent vector comprises third information representing the third image data; transmitting the third latent vector to the pretrained fusor; generating, via the pretrained fusor, a second fused latent vector comprising second fused information, wherein the second fused information comprises the fused information of the fused latent vector, and new information of the third information of the third latent vector, wherein the new information comprises information of the third information that is not in the fused information; transmitting the second fused latent vector to the pretrained decoder.
Clause 9. The computer-implemented method of Clause 8, wherein the third imaging device is the second imaging device.
Clause 10. A system for processing image data during surgery, the system comprising: an imaging device configured to capture image data; a computing device comprising: a memory configured to store computer-executable instructions; and at least one processor in communication with the memory, wherein the computer-executable instructions, when executed by the at least one processor, cause the at least one processor to: receive first image data from the imaging device, wherein the first image data is captured by the imaging device at a first time; generate, via a pretrained encoder, a first latent vector from the first image data, wherein the first latent vector comprises first information representing the first image data; receive second image data from the imaging device, wherein the second image data is captured by the imaging device at a second time, and wherein the second time is after the first time; generate, via the pretrained encoder, a second latent vector from the second image data, wherein the second latent vector comprises second information representing the second image data; transmit the first latent vector and the second latent vector to a pretrained fusor; generate, via the pretrained fusor, a fused latent vector comprising fused information, wherein the fused information comprises the first information of the first latent vector, and new information of the second information of the second latent vector, wherein the new information comprises information of the second information that is not in the first information; transmit the fused latent vector to a pretrained decoder, wherein the pretrained decoder is configured to perform a clinical function from the fused information.
Clause 11. The system of Clause 10, wherein the imaging device comprises scanning laser ophthalmoscopy (SLO), optical coherence tomography (OCT), x-ray, magnetic resonance imaging (MRI), computed tomography scanning (CT), ultrasound, and/or fluorescein angiography (FA).
Clause 12. The system of Clause 10, wherein the first image data and/or the second image data comprise one or more targets and one or more structures of a patient's body.
Clause 13. The system of Clause 12, wherein the pretrained fusor is configured to incorporate information representing the one or more targets in the fused information.
Clause 14. The system of Clause 12, wherein the pretrained fusor is configured to ignore information representing the one or more structures of the patient's body.
Clause 15. The system of Clause 10, wherein the pretrained fusor is configured to not incorporate redundant information in the fused information, wherein redundant information comprises same information of the first information and the second information.
Clause 16. The system of Clause 10, wherein the computer-executable instructions, when executed by the at least one processor, further cause the at least one processor to: receive third image data from the imaging device, wherein the third image data is captured by the third imaging device at a third time, and wherein the third time is after the second time; generate, via the pretrained encoder, a third latent vector from the third image data, wherein the third latent vector comprises third information representing the third image data; transmit the third latent vector to the pretrained fusor; generate, via the pretrained fusor, a second fused latent vector comprising second fused information, wherein the second fused information comprises the fused information of the fused latent vector, and new information of the third information of the third latent vector, wherein the new information comprises information of the third information that is not in the fused information; and transmit the second fused latent vector to the pretrained decoder.
Clause 17. A computer-implemented method for processing target-specific image data during surgery, the method comprising: receiving image data from a first imaging device, wherein the image data is captured by the first imaging device at a first time; segmenting the image data into a plurality of segments, wherein each segment of the plurality of segments comprises image data of a different target; transmitting a first segment of the plurality of segments comprising image data of a first target to a pretrained encoder; generating, via the pretrained encoder, a first latent vector from the first segment, wherein the first latent vector comprises first information representing the image data of the first target; analyzing the first latent vector to determine a corresponding latent vector in a latent vector database, wherein the corresponding latent vector comprises a latent vector generated from a segment of image data captured by the imaging device at a time before the first time, wherein the corresponding latent vector comprises second information representing the first target; generating, via a pretrained fusor, a fused latent vector comprising fused information, wherein the fused information comprises the second information of the corresponding latent vector, and new information of the first information of the first latent vector, wherein the new information comprises information of the first information that is not in the second information; and transmitting the fused latent vector to a pretrained decoder, wherein the pretrained decoder is configured to perform a clinical function from the fused information.
Clause 18. The computer-implemented method of Clause 17, wherein the imaging device comprises scanning laser ophthalmoscopy (SLO), optical coherence tomography (OCT), x-ray, magnetic resonance imaging (MRI), computed tomography scanning (CT), ultrasound, and/or fluorescein angiography (FA).
Clause 19. The computer-implemented method of Clause 17, wherein the first target comprises an indication of a condition.
Clause 20. The computer-implemented method of Clause 17, wherein the first target comprises a floater.
Clause 21. The computer-implemented method of Clause 17, further comprising: transmitting a second segment of the plurality of segments comprising image data of a second target to the pretrained encoder; generating, via the pretrained encoder, a third latent vector from the second segment, wherein the third latent vector comprises third information representing the image data of the second target; analyzing the third latent vector to determine a second corresponding latent vector in the latent vector database, wherein the second corresponding latent vector comprises a latent vector generated from a segment of image data captured by the imaging device at a time before the first time, wherein the corresponding latent vector comprises fourth information representing the second target; generating, via a pretrained fusor, a second fused latent vector comprising second fused information, wherein the second fused information comprises the fourth information of the second corresponding latent vector, and new information of the third information of the third latent vector, wherein the new information comprises information of the third information that is not in the fourth information; and transmitting the second fused latent vector to the pretrained decoder, wherein the decoder is configured to perform a clinical function from the fused information.
Clause 22. The computer-implemented method of Clause 21, wherein first target comprises an indication of a condition.
Clause 23. The computer-implemented method of Clause 21, wherein the first target comprises a floater.
Clause 24. A computer-implemented method for determining completeness of a latent vector, the method comprising: receiving first image data from an imaging device, wherein the first image data is captured by the imaging device at a first time; generating, via a pretrained encoder, a first latent vector from the first image data, wherein the first latent vector comprises first information representing the first image data; receiving, from a latent vector database, a second latent vector, wherein the second latent vector comprises second information representing second image data captured by the imaging device at a second time, wherein the second time is before the first time; transmitting the first latent vector and the second latent vector to a pretrained fusor; generating, via the pretrained fusor, a fused latent vector comprising fused information, wherein the fused information comprises the second information of the second latent vector and new information of the first information of the first latent vector, wherein the new information comprises information of the first information that is not in the second information; analyzing the second latent vector and the fused latent vector to determine a change between the second latent vector and the fused latent vector, wherein the change comprises a numerical representation of an amount of the new information in the fused latent vector; and comparing the change to a predetermined change threshold.
Clause 25. The computer-implemented method of Clause 24, wherein the predetermined change threshold is based on a clinical function the fused latent vector will be used for.
Clause 26. The computer-implemented method of Clause 25, wherein the predetermined change threshold is a first predetermined change threshold when the clinical function is diagnosing a patient with a condition, wherein the predetermined change threshold is a second predetermined change threshold when the clinical function is generating instructions for a surgical or other treatment device, and wherein the second predetermined change threshold is higher than the first predetermined change threshold.
Clause 27. The computer-implemented method of Clause 24, wherein if the change is within the predetermined change threshold, the fused latent vector is complete.
Clause 28. The computer-implemented method of Clause 24, wherein the change is outside of the predetermined change threshold, wherein the computer-implemented method further comprises: receiving third image data from a second imaging device, wherein the third image data is captured by the second imaging device a third time, wherein the third time is after the first time; generating, via the pretrained encoder, a third latent vector from the third image data; transmitting the third latent vector to the pretrained fusor; generating, via the pretrained fusor, a second fused latent vector comprising second fused information, wherein the second fused information comprises the fused information of the fused latent vector and second new information of the third information of the third latent vector, wherein the second new information comprises information of the third information that is not in the fused information; analyzing the fused latent vector and the second fused latent vector to determine a second change between the fused latent vector and the second fused latent vector, wherein the second change comprises a numerical representation of an amount of the second new information in the second fused latent vector; and comparing the second change to the predetermined change threshold.
Clause 29. The computer-implemented method of Clause 26, wherein the second imaging device is the imaging device.

### Additional Embodiments

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or operations. Thus, such conditional language is not generally intended to imply that features, elements and/or operations are in any way required for one or more embodiments or that one or more embodiments necessarily include logic for deciding, with or without author input or prompting, whether these features, elements and/or operations are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment disclosed herein can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A computer-implemented method for fused latent vector processing, the computer-implemented method comprising:
receiving, by a computer system, first data from a first data source;
receiving, by the computer system, second data from a second data source;
generating, via a first pretrained encoder, a first latent vector from the first data, wherein the first latent vector comprises first information representing the first data;
generating, via a second pretrained encoder, a second latent vector from the second data, wherein the second latent vector comprises second information representing the second data;
generating, via a pretrained fusor, a fused latent vector comprising fused information, wherein the fused information comprises information representing the first information and the second information; and
decoding the fused latent vector using a pretrained decoder, wherein the pretrained decoder is configured to generate an output for use in performing a clinical function.

2. The computer-implemented method of claim 1, wherein the first data source comprises image data from a first imaging device configured to capture image data using a first imagine device configure to capture image data using a first imaging modality, and wherein the second data source comprises image data from a second imaging device configured to capture image data using a second imaging modality.

3. The computer-implemented method of claim 2, wherein the first imaging device and the second imaging device are a same device,
wherein the first imaging modality and the second imaging modality comprise a same imaging modality.

4. The computer-implemented method of claim 2, wherein the first imaging modality and the second imaging modality comprise at least one of: optical coherence tomography (OCT), scanning laser ophthalmoscopy (SLO), ultrasound, computed tomography (CT), x-ray, magnetic resonance imaging (MRI), or fluorescein angiography (FA).

5. The computer-implemented method of claim 1, wherein the pretrained fusor is configured to not incorporate redundant information in the fused information, wherein redundant information comprises same information of the first information and the second information.

6. The computer-implemented method of claim 1, further comprising:
receiving, by the computer system, third data from a third data source;
generating, via a third pretrained encoder, a third latent vector from the third data, wherein the third latent vector comprises third information representing the third data; and
generating, via the pretrained fusor, a second fused latent vector comprising second fused information, wherein the pretrained fusor is configured to merge the fused latent vector and the third latent vector to generate the second fused latent vector.

7. The computer-implemented method of claim 6, further comprising:
determining a completeness of the second fused latent vector, wherein determining the completeness of the second fused latent vector comprises:
determining a similarity between the fused latent vector and the second fused latent vector; and
comparing the similarity to a similarity threshold, wherein second fused latent vector is determined to be incomplete when the similarity is below the similarity threshold.

8. The computer-implemented method of claim 1, wherein the first data comprises a first OCT sample at a first location in a field of view,
wherein the second data comprises a second OCT sample at a second location in the field of view, and
wherein the pretrained decoder is configured to generate an OCT image of the field of view.

9. The computer-implemented method of claim 1, wherein the first data comprises an OCT sample at a first location in a field of view,
wherein the second data comprises an SLO image of the field of view, and
wherein the pretrained decoder is configured to generate an OCT image of the field of view.

10. The computer-implemented method of claim 1, wherein the first data comprises a first OCT sample at a first location in a field of view,
wherein the second data comprises a second OCT sample at a second location in the field of view, and
wherein the pretrained decoder is configured to be used to generate a treatment plan, the treatment plan defining one or more treatment locations for treatment of a floater with a laser.

11. A system for fused latent vector processing comprising:
at least one processor; and
a computer-readable, non-volatile storage medium having instructions stored thereon that, when executed by the at least one processor, cause the system to:
receive first data from a first data source;
receive second data from a second data source;
generate, via a first pretrained encoder, a first latent vector from the first data, wherein the first latent vector comprises first information representing the first data;
generate, via a second pretrained encoder, a second latent vector from the second data, wherein the second latent vector comprises second information representing the second data;
generate, via a pretrained fusor, a fused latent vector comprising fused information, wherein the fused information comprises information representing the first information and the second information; and
decode the fused latent vector using a pretrained decoder, wherein the pretrained decoder is configured to generate an output for use in performing a clinical function.

12. The system of claim 11, wherein the first data source comprises image data from a first imaging device configured to capture image data using a first imagine device configure to capture image data using a first imaging modality, and wherein the second data source comprises image data from a second imaging device configured to capture image data using a second imaging modality.

13. The system of claim 12, wherein the first imaging device and the second imaging device are a same device,
wherein the first imaging modality and the second imaging modality comprise a same imaging modality.

14. The system of claim 12, wherein the first imaging modality and the second imaging modality comprise at least one of: optical coherence tomography (OCT), scanning laser ophthalmoscopy (SLO), ultrasound, computed tomography (CT), x-ray, magnetic resonance imaging (MRI), or fluorescein angiography (FA).

15. The system of claim 11, wherein the pretrained fusor is configured to not incorporate redundant information in the fused information, wherein redundant information comprises same information of the first information and the second information.

16. The system of claim 11, wherein the instructions are further configured to\cause the system to:
receive third data from a third data source;
generate, via a third pretrained encoder, a third latent vector from the third data, wherein the third latent vector comprises third information representing the third data; and
generate, via the pretrained fusor, a second fused latent vector comprising second fused information, wherein the pretrained fusor is configured to merge the fused latent vector and the third latent vector to generate the second fused latent vector.

17. The system of claim 16, wherein the instructions are further configured to cause the system to:
determine a completeness of the second fused latent vector, wherein determining the completeness of the second fused latent vector comprises:
determining a similarity between the fused latent vector and the second fused latent vector; and
comparing the similarity to a similarity threshold, wherein second fused latent vector is determined to be incomplete when the similarity is below the similarity threshold.

18. The system of claim 11, wherein the first data comprises a first OCT sample at a first location in a field of view,
wherein the second data comprises a second OCT sample at a second location in the field of view, and
wherein the pretrained decoder is configured to generate an OCT image of the field of view.

19. The system of claim 11, wherein the first data comprises an OCT sample at a first location in a field of view,
wherein the second data comprises an SLO image of the field of view, and
wherein the pretrained decoder is configured to generate an OCT image of the field of view.

20. The system of claim 11, wherein the first data comprises a first OCT sample at a first location in a field of view,
wherein the second data comprises a second OCT sample at a second location in the field of view, and
wherein the pretrained decoder is configured to be used to generate a treatment plan, the treatment plan defining one or more treatment locations for treatment of a floater with a laser.
